(19) United States
(12) Patent Application Publication
(10) Pub. No.: US 2024/0180978 A1
(43) Pub. Date: Jun. 6, 2024

(54) PREPARATIONS COMPRISING PROBIOTIC STRAINS AND L-TRYPTOPHAN

(71) Applicant: EVONIK OPERATIONS GMBH, Essen (DE)

(72) Inventors: Bodo SPECKMANN, Kahl (DE); Michael SCHWARM, Alzenau (DE); Michael SCHILLING, Berlin (DE)

(73) Assignee: EVONIK OPERATIONS GMBH, Essen (DE)

(21) Appl. No.: 18/279,388

(22) PCT Filed: Feb. 28, 2022
(86) PCT No.: PCT/EP2022/054979

§ 371 Date: Aug. 30, 2023

(30) Foreign Application Priority Data
Mar. 1, 2021 (EP) 21159973.3

(51) Int. Cl. A61K 35/747; A23L 33/135; A23L 33/175; A61K 31/405; C12N 1/20; C12R 1/24; C12R 1/25

(52) U.S. Cl. CPC A61K 35/747; A23L 33/135; A23L 33/175; A61K 31/405; C12N 1/20; C12R 2001/24; C12R 2001/25

(57) ABSTRACT

The current invention concerns preparations comprising probiotic strains belonging to the genus Lactobacillus in combination with the amino acid L-tryptophan.

The current invention concerns preparations comprising at least one probiotic strain belonging to the species Lactobacillus plantarum (Lactiplantibacillus plantarum), Lactobacillus hilgardii (Lentilactobacillus hilgardii), Lactobacillus paracasei (Lacticaseibacillus paracasei), Lactobacillus brevis, Lactobacillus delbrueckii, Lactobacillus crispatus, Lactobacillus reuteri (Limosilactobacillus reuteri), and sources of L-tryptophan in a colon-release formulation.

Nutrition is an important contributor to the health of humans and animals. The gastrointestinal microbiota acts as a relevant mediator of nutrient—as well as active pharmaceutical ingredient-triggered health effects and has therefore emerged as a target of interventions to improve health. Microbiota-targeted strategies include the application of prebiotics, probiotics and synbiotics to modulate the microbiota's composition and activity. A host-centered definition of prebiotic describes it as “a substrate that is selectively utilized by host microorganisms conferring a health benefit” (Consensus definition by the International Scientific Association for Probiotics and Prebiotics (ISAPP)) [1], thus referring not only to certain carbohydrates but also to e.g. amino acids and peptides as prebiotics. Probiotics are defined as: “live microorganisms that, when administered in adequate amounts, confer a health benefit on the host” (ISAPP definition) [2]. The most commonly investigated and commercially available probiotics are mainly microorganisms from species of genera Lactobacillus and Bifidobacterium. In addition, several others such as Propionibacterium, Streptococcus, Bacillus, Enterococcus, Escherichia coli, and yeasts are also used. Synbiotics refer to food ingredients or supplements combining probiotics and prebiotics in a form of synergism, hence synbiotic [3]. In the context of this invention, we understand the term “synbiotics” as combinations of probiotics with any chemically defined substance(/s), e.g. amino acids, peptides, fatty acids, and carbohydrates.

L-tryptophan (Trp) is a proteinogenic amino acid and as such used for protein biosynthesis; besides this Trp is metabolized to compounds such as nicotinic acid mononucleotide, nicotinamide dinucleotide, and serotonin, each of which has specific biochemical functions and thereby affects physiology. Trp enters the body via normal dietary ingestion of Trp-containing proteins included in food stuffs. Additionally, Trp as amino acid can also be ingested in the form of dietary supplements.

Mammalian non-proteinogenic Trp metabolism is executed by host as well as by select microbial cells; gut microbial Trp metabolism thereby contributes to the fecal as well as the circulating pool of Trp metabolites in mammals [4]. Of note, some Trp metabolites, including indole-3 lactic acid (ILA) and indole-3 acetic acid (IAA), appear to be exclusively produced by gut microbes and not by host cells [5]. In line with this, fecal levels of IAA and L-kynurenine are drastically reduced in germ-free compared to conventional mice [5]. Gut microbiota composition and Trp availability are therefore major determinants of bioavailability of these compounds.

Health effects of Trp metabolites are inferred by mechanistic studies, animal studies and by human association studies. Kynurenine and IAA are linked to psychological functions such as mood, appetite, and anxiety, supposedly via effects on neuroinflammation as well as Trp uptake via the blood-brain-barrier, and Trp-to-serotonin metabolization [6, 7]. Kynurenine promotes expansion of gut mucosal RORγt (+)IL-22(+) ILC3 cells, which in turn stimulate proliferation of mucus-producing goblet cells and thereby support gut barrier integrity [8]. ILA has been described to protect against inflammatory bowel diseases through modulation of mucosal CD4+ T-cell differentiation [9]. Some Trp metabolites are agonists of the arylhydrocarbon receptor (AhR) [10-12], a transcription factor that regulates the expression of genes involved in xenobiotics metabolism [13], immunity [9], the expression of interleukin-22 [11], in various organs, including the liver, gut, lung, and brain. AhR thereby affects various health conditions, e.g. chronic-inflammatory diseases of the gut (colitis), lung (e.g. asthma bronchiale), and brain (e.g. major depressive disorder). Other Trp metabolites like indole-3-propionic acid reportedly engage the pregnane-X receptor and thereby regulate intestinal barrier function [14].

Translation of these preclinical findings towards improving human health has however shown to be challenging. Direct delivery of Trp metabolites by intravenous injection is not feasible for humans, particularly not in the context of preventive approaches. We reason that the conversion of Trp to Trp metabolites is a crucial step which is decisive for delivering successful outcomes from any interventions aiming to prevent, cure, or treat health conditions with Trp. We also conceive that the Trp-metabolizing machinery of the gut microbiome is dysfunctional under certain conditions, e.g. under a high-salt diet [15].

The objective of this invention is therefore to provide a technology that promotes the beneficial metabolization of highly available Trp by potent gut microbes inside an organism to provide a benefit for humans and animals suffering from the above-mentioned conditions and that are in need of novel strategies to prevent, ameliorate or cure such and similar conditions.

This goal is achieved by the invention combining a suitable Trp source with potent microbial Trp-metabolizers in a suitable colonic-release formulation.

Recently, the taxonomic classification of several species of the genus Lactobacillus has been updated, according to Zheng J, Wittouck S, Salvetti E, Cmap Franz H M B, Harris P, Mattarelli P W, O'Toole B, Pot P, Vandamme J, Walter K, Watanabe S, Wuyts G E, Felis M G, Ganzle A and Lebeer S, 2020. A taxonomic note on the genus lactobacillus: description of 23 novel genera, emended description of the genus lactobacillus Beijerinck 1901, and Union of Lactobacilliae and Leuconostocaceae. International Journal of Systematic and Evolutionary Microbiology. https://doi.org/10.1099/ijsem.0.004107. Of particular relevance in the context of this invention are the following species:

| “Old” denomination | Updated denomination (since 2020) |
|---|---|
| Lactobacillus brevis | Levilactobacillus brevis |
| Lactobacillus crispatus | Lactobacillus crispatus |
| Lactobacillus delbrueckii | Lactobacillus delbrueckii |
| Lactobacillus hilgardii | Lentilactobacillus hilgardii |
| Lactobacillus paracasei | Lacticaseibacillus paracasei |
| Lactobacillus plantarum | Lactiplantibacillus plantarum |
| Lactobacillus reuteri | Limosilactobacillus reuteri |

WO16077190A1 discloses a pharmaceutical composition comprising composite particles comprising Lactobacillus and Trp used as an excipient.

US 2015/0258151 A1 discloses methods for administering Clostridium sporogenes or E. coli bacteria that produce select metabolites of tryptophan (including IAA) to humans for use in treatment and prevention of gut barrier dysfunction in humans, without disclosing an amount of IAA that is produced by such bacteria, or the use of a colon-targeting formulation.

Zelante et al. reported production of ILA and IAA by a Lactobacillus acidophilus, albeit at very low levels [11], whereas Wilck et al. proposed Lactobacillus murinus as a direct or indirect source of gut microbiota-derived ILA and IAA [15].

Cervantes-Baragan et al. disclose combinations of Lactobacillus reuteri (Limosilactobacillus reuteri) and a Trp-rich diet to stimulate the formation of regulatory T cells in the gastrointestinal mucosa [9].

Despite these indications for few Lactobacillus sp., Clostridium sporogenes, and E. coli being a source of some Trp metabolites, a comparative and quantitative analysis of the Trp metabolome of relevant probiotic Lactobacillus sp. has not been described.

We reasoned that such an analysis is the prerequisite to unlock the potential of targeted formation of selected Trp metabolites by the gut microbiota to benefit the host in the prevention and/or curing of targetable diseases. Moreover, we disclose a technical solution for achieving this goal through preparations comprising sources of Trp together with Lactobacillus strains with unprecedented capacity to produce KYN, ILA, and/or IAA from this added Trp in formulations that facilitate a targeted release in the distal parts of the gastrointestinal tract (jejunum, large bowel, distal colon), whereby these formulations enable the biosynthesis of these substances by detaining Trp from its absorption in the upper parts of the small intestine. Furthermore, the Lactobacillus strains that we have discovered display surprisingly rapid conversion of Trp towards KYN, ILA, and/or IAA and thus have a competitive advantage against other members of the microbiota or absorption through host cells.

The present invention is directed to a preparation comprising at least one probiotic strain belonging to the species Lactobacillus paracasei (Lacticaseibacillus paracasei), Lactobacillus brevis (Levilactobacillus brevis), Lactobacillus delbrueckii, Lactobacillus crispatus, Lactobacillus plantarum (Lactiplantibacillus plantarum, Lactobacillus plantarum subspecies argentoratensis, Lactobacillus reuteri (Limosilactobacillus reuteri), and Lactobacillus hilgardii (Lentilactobacillus hilgardii) and L-tryptophan or a dipeptide containing L-tryptophan or a foodstuff, fruit or plant or meat extract containing L-tryptophan.

This new preparation promotes unparalleled levels of beneficial Trp metabolites in the large intestinal lumen, whereby they become available to the host and exert physiological functions therein.

In a preferred embodiment, Trp is either in the form of free Trp or contained in dipeptides or in a chemically modified form of Trp, e.g. N-Acetyl-Trp.

It is preferred, when the L-tryptophan is in a foodstuff, fruit or plant or meat extract, and L-tryptophan is present in the foodstuff, fruit or plant or meat extract at a concentration of at least 0.01 weight-%, preferably at least 0.10 weight-% and the foodstuff, fruit or plant or meat extract is preferably selected from soy beans, cashew nuts, peanuts, lentils, oat, quark, egg, tuna, chicken.

Another aspect of the invention is directed to a preparation which further comprising a targeted-release formulation for delayed release or enteric or colonic release. A targeted-release formulation according to the present invention is a formulation which ensures the delivery of the component of the preparation according to the present invention to a specific target in the body. A preferred formulation of such preparations promotes enteral or colonic delivery in the lower small intestine or in the large intestine. The targeted-release formulation can be obtained by adding enteric polymers to the matrix of the dosage form, or by adding a coating to the dosage form, preferably an enteric coating.

According to the present invention, a colon-specific delivery system is a delivery system, which targets the substance or drug directly to the colon. The advantage of a colon-specific delivery system is the local action, in case of disorders like ulcerative colitis, Crohn's disease, irritable bowel syndrome, and carcinomas. Targeted drug delivery to the colon in these cases ensures direct treatment at the site with lower dosing and fewer systemic side effects. In addition to local therapy colon can also be utilized as the portal entry of the drugs into systemic circulation for example molecules that are degraded/poorly absorbed in upper gut such as proteins and peptides may be better absorbed from the more benign environment of the colon. Colon-specific drug delivery is considered beneficial in the treatment of colon-related diseases and the oral delivery of protein and peptide drugs. Generally, each colon-specific drug delivery system has been designed based on one of the following mechanisms with varying degrees of success; 1. Coating with pH dependent polymers, 2. Coating with pH independent biodegradable polymers and 3. Delivery systems based on the metabolic activity of colonic bacteria.

An enteric coating is a barrier applied on oral medication that prevents its dissolution or disintegration in the gastric environment. Most enteric coatings work by presenting a surface that is stable at the intensely acidic pH found in the stomach but breaks down rapidly at a higher pH (alkaline pH). For example, they will not dissolve in the gastric acids of the stomach (pH ˜3), but they will start to dissolve in the environment present in the distal small intestine (pH range proximal to distal small intestine is ˜5.6 to 7.4) [11]. Colon targeted (drug) delivery systems are designed to selectively release a drug in response to the colonic environment without premature drug release in the upper GI tract.

The colon-specific delivery system can comprise a pH-dependent drug delivery system, since the colon exhibits a relatively higher pH than the upper GI tract. Accordingly, a colon-targeted delivery system is designed by using pH-dependent polymers such as cellulose acetate phthalates (CAP), hydroxypropyl methyl-cellulose phthalate (HPMCP) 50 and 55, copolymers of methacrylic acid and methyl methacrylate (e.g., Eudragit® S 100, Eudragit® L, Eudragit® FS, and Eudragit® P4135 F).

Therefore, in an advantageous configuration, the colon-specific delivery system comprises a coating comprising at least one pH dependent polymer or biodegradable polymer, preferably selected from methyl acrylate-methacrylic acid copolymers, cellulose acetate phthalate (CAP), cellulose acetate succinate, hydroxypropyl methyl cellulose phthalate, hydroxypropyl methyl cellulose acetate succinate (hypromellose acetate succinate), polyvinyl acetate phthalate (PVAP), methyl methacrylate-methacrylic acid copolymers, shellac, cellulose acetate trimellitate, sodium alginate, zein.

As a coating it is preferred to use a polymer polymerized from 10 to 30% by weight methyl methacrylate, 50 to 70% by weight methyl acrylate and 5 to 15% by weight methacrylic acid.

The polymer dispersion as disclosed may preferably comprise 15 to 50% by weight of a polymer polymerized from 20 to 30% by weight methyl methacrylate, 60 to 70% by weight methyl acrylate and 8 to 12% by weight methacrylic acid. Most preferred the polymer is polymerized from 25% by weight methyl methacrylate, 65% by weight methyl acrylate and 10% by weight methacrylic acid.

A 30% by weight aqueous dispersion of a polymer polymerized from 25% by weight methyl methacrylate, 65% by weight methyl acrylate and 10% by weight methacrylic acid corresponds to the commercial product EUDRAGUARD® biotic.

The percentages of the monomers add up to 100%. The functional polymer is applied in amounts of 2-30 mg/cm², preferably 5-20 mg/cm².

In a preferred embodiment the probiotic strain is selected from Lactobacillus plantarum (Lactiplantibacillus plantarum DSM 33447, Lactobacillus delbrueckii DSM 33431, Lactobacillus brevis (Levilactobacillus brevis) DSM 33429, Lactobacillus plantarum subspecies argentoratensis DSM 33449. In a further preferred configuration of the present invention, the preparation comprises two or more of the listed probiotic strains, more preferred three or more of the probiotic strains and particularly preferred all the probiotic strains listed above.

Above-mentioned strains have been identified by screening of naturally occurring isolates. They have been deposited at the Leibniz-Institut DSMZ Deutsche Sammlung von Mikroorganismen and Zellkulturen GmbH, Inhoffenstr. 7B, 38124 Braunschweig, Germany under the provisions of the Budapest Treaty on the International Recognition of the Deposit of Microorganisms for the Purpose of Patent Procedure under the Accession Numbers Lactobacillus plantarum (Lactiplantibacillus plantarum DSM 33447, Lactobacillus delbrueckii DSM 33431, Lactobacillus brevis (Levilactobacillus brevis) DSM 33429, Lactobacillus plantarum subspecies argentoratensis DSM 33449 in the name of Novozymes Berlin GmbH, Gustav-Meyer-Allee 25, 13355 Berlin, Germany.

Thus, the Lactobacillus strains used for the preparations according to the present invention is selected from the following group:
a) The strains as deposited under DSM 33447, DSM 33431, DSM 33429, DSM 33449 at the DSMZ;
b) mutants of the strains as deposited under DSM 33447, DSM 33431, DSM 33429, DSM 33449 having all identifying characteristics of the strains DSM 33447, DSM 33431, DSM 33429, DSM 33449, wherein said mutant preferably has a DNA sequence identity to the strains DSM 33447, DSM 33431, DSM 33429, DSM 33449 of at least 95%, preferably at least 96, 97 or 98%, more preferably at least 99 or 99.5%;
c) a preparation of (a) or (b);
d) a preparation containing an effective mixture of metabolites as contained in (a), (b) or (c).

In a preferred embodiment, Lactobacillus plantarum (Lactiplantibacillus plantarum) or the specific Lactobacillus plantarum (Lactiplantibacillus plantarum) DSM 33447 strain exhibits the following characterizing sequences:
a) A groL sequence with a sequence identity of at least 95%, more preferably 99.5%, most preferably 100%, to the polynucleotide sequence according to SEQ ID NO: 1;
b) A gyrB sequence with a sequence identity of at least 95%, more preferably 99.5%, most preferably 100%, to the polynucleotide sequence according to SEQ ID NO: 2;
c) A dnaA sequence with a sequence identity of at least 95%, more preferably 99.5%, most preferably 100%, to the polynucleotide sequence according to SEQ ID NO: 3;
d) A rpsK sequence with a sequence identity of at least 95%, more preferably 99.5%, most preferably 100%, to the polynucleotide sequence according to SEQ ID NO: 4;
e) A rpmB sequence with a sequence identity of at least 95%, more preferably 99.5%, most preferably 100%, to the polynucleotide sequence according to SEQ ID NO: 5;
f) A consensus 16 rDNA sequence with a sequence identity of at least 95%, more preferably 99.5%, most preferably 100%, to the polynucleotide sequence according to SEQ ID NO: 6.

In a preferred configuration, Lactobacillus delbrueckii or the specific Lactobacillus delbrueckii DSM 33431 strain exhibits the following characterizing sequences:
a) A groL sequence with a sequence identity of at least 95%, more preferably 99.5%, most preferably 100%, to the polynucleotide sequence according to SEQ ID NO: 7;
b) A gyrB sequence with a sequence identity of at least 95%, more preferably 99.5%, most preferably 100%, to the polynucleotide sequence according to SEQ ID NO: 8;
c) A dnaA sequence with a sequence identity of at least 95%, more preferably 99.5%, most preferably 100%, to the polynucleotide sequence according to SEQ ID NO: 9;
d) A rpsK sequence with a sequence identity of at least 95%, more preferably 99.5%, most preferably 100%, to the polynucleotide sequence according to SEQ ID NO: 10;
e) A rpmB sequence with a sequence identity of at least 95%, more preferably 99.5%, most preferably 100%, to the polynucleotide sequence according to SEQ ID NO: 11;
f) A consensus 16 rDNA sequence with a sequence identity of at least 95%, more preferably 99.5%, most preferably 100%, to the polynucleotide sequence according to SEQ ID NO: 12.

In a preferred configuration, Lactobacillus brevis (Levilactobacillus brevis) or the specific Lactobacillus brevis (Levilactobacillus brevis) strain DSM 33429 exhibits the following characterizing sequences:
a) A groL sequence with a sequence identity of at least 95%, more preferably 99.5%, most preferably 100%, to the polynucleotide sequence according to SEQ ID NO: 13;
b) A gyrB sequence with a sequence identity of at least 95%, more preferably 99.5%, most preferably 100%, to the polynucleotide sequence according to SEQ ID NO: 14;
c) A dnaA sequence with a sequence identity of at least 95%, more preferably 99.5%, most preferably 100%, to the polynucleotide sequence according to SEQ ID NO: 15;
d) A rpsK sequence with a sequence identity of at least 95%, more preferably 99.5%, most preferably 100%, to the polynucleotide sequence according to SEQ ID NO: 16;
e) A rpmB sequence with a sequence identity of at least 95%, more preferably 99.5%, most preferably 100%, to the polynucleotide sequence according to SEQ ID NO: 17;
f) A consensus 16 rDNA sequence with a sequence identity of at least 95%, more preferably 99.5%, most preferably 100%, to the polynucleotide sequence according to SEQ ID NO: 18.

In a preferred configuration, Lactobacillus plantarum subspecies argentoratensis or the specific Lactobacillus plantarum subspecies argentoratensis DSM 33449 strain exhibits the following characterizing sequences:
a) A groL sequence with a sequence identity of at least 95%, more preferably 99.5%, most preferably 100%, to the polynucleotide sequence according to SEQ ID NO: 19;
b) A gyrB sequence with a sequence identity of at least 95%, more preferably 99.5%, most preferably 100%, to the polynucleotide sequence according to SEQ ID NO: 20;
c) A dnaA sequence with a sequence identity of at least 95%, more preferably 99.5%, most preferably 100%, to the polynucleotide sequence according to SEQ ID NO: 21;
d) A rpsK sequence with a sequence identity of at least 95%, more preferably 99.5%, most preferably 100%, to the polynucleotide sequence according to SEQ ID NO: 22;
e) A rpmB sequence with a sequence identity of at least 95%, more preferably 99.5%, most preferably 100%, to the polynucleotide sequence according to SEQ ID NO: 23;
f) A consensus 16 rDNA sequence with a sequence identity of at least 95%, more preferably 99.5%, most preferably 100%, to the polynucleotide sequence according to SEQ ID NO: 24.

Thus, a further subject of the current invention is a Lactobacillus strain, in particular a Lactobacillus strain as mentioned before, exhibiting at least one, preferably all of the following characteristics:
a) a groL sequence with a sequence identity of at least 95%, or 96%, or 97%, or 98%, or 99%, preferably at least 99.5%, more preferably at least 99.8 or 99.9%, above all 100%, to the polynucleotide sequence according to SEQ ID NO: 1 or SEQ ID NO: 7 or SEQ ID NO: 13 or SEQ ID NO: 19;
c) a gyrB sequence with a sequence identity of at least 95%, or 96%, or 97%, or 98%, or 99%, preferably at least 99.5%, more preferably at least 99.8 or 99.9%, above all 100%, to the polynucleotide sequence according to SEQ ID NO: 2 or SEQ ID NO: 8 or SEQ ID NO: 14 or SEQ ID NO: 20;
d) a dnaA sequence with a sequence identity of at least 95%, or 96%, or 97%, or 98%, or 99%, preferably at least 99.5%, more preferably at least 99.8 or 99.9%, above all 100%, to the polynucleotide sequence according to SEQ ID NO: 3 or SEQ ID NO: 9 or SEQ ID NO: 15 or SEQ ID NO: 21;
e) a rpsK sequence with a sequence identity of at least 95%, or 96%, or 97%, or 98%, or 99%, preferably at least 99.5%, more preferably at least 99.8 or 99.9%, above all 100%, to the polynucleotide sequence according to SEQ ID NO: 4 or SEQ ID NO: 10 or SEQ ID NO: 16 or SEQ ID NO: 22;
f) a rpmB sequence with a sequence identity of at least 95%, or 96%, or 97%, or 98%, or 99%, preferably at least 99.5%, more preferably at least 99.8 or 99.9%, above all 100%, to the polynucleotide sequence according to SEQ ID NO: 5 or SEQ ID NO: 11 or SEQ ID NO: 17 or SEQ ID NO: 23;
g) a 16S rDNA consensus sequence with a sequence identity of at least 95%, or 96%, or 97%, or 98%, or 99%, preferably at least 99.5%, more preferably at least 99.8 or 99.9%, above all 100%, to the polynucleotide sequence according to SEQ ID NO: 6 or SEQ ID NO: 12 or SEQ ID NO: 18 or SEQ ID NO: 24.

In a further preferred configuration, the preparation according to the present invention in a minimum medium with a carbohydrate concentration of not more than 5 g/l one or more of the following metabolites are produced: indole-3 lactic acid, indole-3 acetic acid, and L-kynurenine, preferably in amounts of at least 3 mg/l indole-3 lactic acid, 60 μg/l indole-3 acetic acid, and 20 μg/l L-kynurenine.

It is further preferred, when the probiotic strain is present in a dose range of 1×10⁷-1×10¹¹ colony-forming units (CFU).

In an advantageous configuration, L-tryptophan is present in an amount of at least 10 mg, preferably at least 50 mg, more preferably at least 100 mg.

The preparation may further contain further carbohydrate ingredients, selected from arabinoxylans, barley grain fibre, oat grain fibre, rye fibre, wheat bran fibre, inulins, fructooligosaccharides (FOS), galactooligosaccharides (GOS), resistant starch, beta-glucans, glucomannans, galactoglucomannans, guar gum and xylooligosaccharides.

The preparation may further contain one or more plant extracts, selected from valerian root, ashwagandha, saint john's wort, rose of Sharon, hop, ginger, cinnamon, grapefruit, parsley, turmeric, curcuma, olive fruit, panax ginseng, horseradish, garlic, broccoli, spirulina, pomegranate, cauliflower, kale, cilantro, green tea, onions, and milk thistle.

The preparation may comprise further vitamins or co-factors selected from biotin, vitamin A, vitamin B1 (thiamine), vitamin B2 (riboflavin), vitamin B3 (niacin), vitamin B5 (pantothenic acid), vitamin B6 (pyridoxin, pyridoxal), vitamin B9 (folic acid or folate), vitamin C (ascorbic acid), vitamin D (calciferols), vitamin E (tocopherols and tocotrienols) and vitamin K (quinones), S-adenosyl methionine, cysteine, N-acetyl cysteine, or minerals selected from sulfur, iron, chlorine, calcium, chromium, cobalt, copper, magnesium, manganese, molybdenum, iodine, selenium, and zinc.

The preparation may further contain astaxanthin, charcoal, chitosan, glutathione, monacolin K, plant sterols, plant stanols, sulforaphane, collagen, hyaluronic acid, phosphatidylcholine, eicosapentaenoic acid (EPA), docosahexaenoic acid (DHA), melatonin, diphenhydramin One subject of the present invention is the use of a preparation according to the present invention as food supplement or its use in foodstuffs. Preferred foodstuffs according to the invention are chocolate and cocoa products, gummies, mueslis, muesli bars, dairy products, breads, and pastries.

A further subject of the current invention is also the use of a preparation of the current invention as a synbiotic ingredient in food products.

A further subject of the present invention is foodstuff composition containing a preparation according to the present invention and at least one further feed or food ingredient, preferably selected from proteins, carbohydrates, fats, further probiotics, prebiotics, enzymes, vitamins, immune modulators, milk replacers, minerals, amino acids, coccidiostats, acid-based products, medicines, and combinations thereof.

The foodstuff composition according to the present invention does also include dietary supplements in the form of a pill, capsule, tablet, straw, or liquid.

The preparations according to the present invention, when administered to human beings, preferably improve the health status, in particular mental health, sleep, gut health, immune health, healthy weight of a human being.

A further subject of the current invention is therefore a composition according to the present invention for improving the health status, in particular mental health, sleep, gut health, immune health, healthy weight of a human being.

An advantageous configuration according to the present invention is a composition for improving the health status of an animal or a human being by one or more of the following:
increasing the total amount of the following Trp metabolites in the host via their production by gastrointestinal microorganisms: Indole-3 acetic acid (CAS 6505-45-9), Indole-3 lactic acid (CAS 1821-52-9), L-kynurenine (CAS 2922-83-0).
increasing the bioavailability of L-tryptophan in the central nervous system
increasing the biosynthesis of L-serotonin in the central nervous system
increasing the biosynthesis of melatonin in the central nervous system
decreasing neuroinflammation

Another aspect of the present invention is directed to the use of a preparation according to the present invention as food supplement.

In a further preferred configuration of the use of the preparation according to the present invention in a minimum medium with a carbohydrate concentration of not more than 5 g/l one or more of the following metabolites are produced: indole-3 lactic acid, indole-3 acetic acid, and L-kynurenine, preferably in amounts of at least 3 mg/l indole-3 lactic acid, 60 μg/l indole-3 acetic acid, and 20 μg/L-kynurenine.

### WORKING EXAMPLES

### Example 1: The Production of Trp Metabolites by Lactobacillus Strains from Trp Under Different Culture Conditions

In order to select an appropriate medium for production of relevant metabolites according to the invention, 88 randomly selected Lactobacillus strains were cultivated under anaerobic conditions in microtiter plates with MRS medium for 48 h at 37° C. Afterwards the cells were harvested by centrifugation at 4000×g for 10 min and washed with PBS buffer (containing 1.5 g/l Na₂HPO₄*2H₂O, 0.2 g/l KH₂PO₄ and 8.8 g/l NaCl, the pH value was adjusted with HCl to 7.0). Subsequently the cells were resuspended in media listed in table 1 containing 0.8 mM of L-tryptophan as substrate and incubated for 6 h. Afterwards the cells were separated by centrifugation and the supernatant was subjected to HPLC analysis. The aim was to identify a medium which supports the biosynthesis of relevant compounds. The results are shown in table 2.

TABLE 1
Media tested for the biosynthesis of relevant compounds,

| Medium^{a} | Components |
|---|---|
| Peptone- | 10 g/l tryptone^{b}, 5 g/l NaCl |
| water | |
| M9- | 2 mM MgSO₄, 0.1 mM CaCl₂, 42.8 mM Na₂HPO₄, |
| medium | 22 mM KH₂PO₄, 8.4 mM NaCl, 18.5 mM NH₄Cl, |
| | 0.2% glucose; pH = 7.4 |
| FeSSIF | 15 mM Na-taurocholate, 3.75 mM lecithin, |
| | 203 mM NaCL, 101 mM NaOH, 144 mM acetic |
| | acid; pH = 5 |

^{a}All media were supplemented with L-tryptophan to a final concentration of 0.8 mM,
^{b}contains approx. 1% (w/w) L tryptophan (≈0.5 mM)

TABLE 2
Production of relevant compounds from L-tryptophan in different
media. Detection of compounds was performed with HPLC

| | Media tested | | |
|---|---|---|---|
| No. of strains producing | M9-Medium | FeSSIF | Peptone Medium |
| Indole | 0 | 0 | 0 |
| Indole-3-acetamide | 0 | 0 | 0 |
| Indole-3-pyruvic acid | 0 | 0 | 0 |
| Indole-3-lactic acid | 24 | 0 | 0 |
| Indole-3-acetic acid | 3 | 0 | 0 |
| Serotonine | 0 | 0 | 0 |
| Kynurenine | 0 | 0 | 0 |

The result shown in table 2 clearly demonstrate that the different strains were only able to produce relevant compounds in M9 medium. In this case 24 out of 88 strains were able to produce at least one of the desired compounds. In general, the most abundant compounds were indole-3-lactic acid and indole-3-aldehyde. Based on these results M9-medium was selected for the screening.

### Example 2: Lactobacillus Species Display Varying Potential of Producing Trp Metabolites

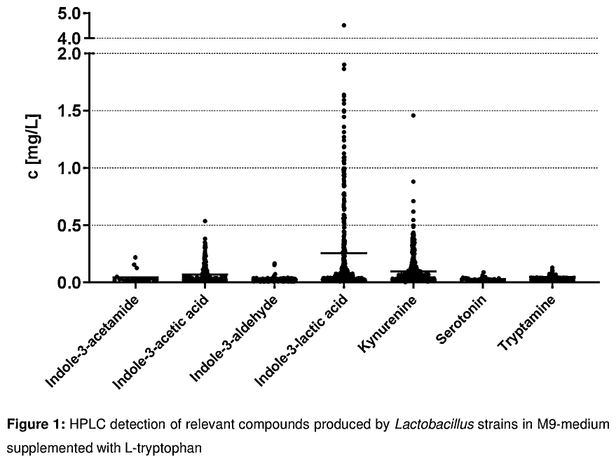

600 different Lactobacillus strains were cultivated under anaerobic conditions in microtiter plates with MRS medium for 48 h at 37° C. Afterwards the cells were harvested by centrifugation at 4000×g for 10 min and washed with PBS buffer. Subsequently the cells were resuspended in M9-medium supplemented with 0.8 mM L-tryptophan and transferred to deep-well plates. After 6 h incubation under anaerobic conditions at 37° C., the cells were removed by centrifugation at 4000×g for 10 min. Product formation was determined by HPLC analysis of the supernatant. The results are summarized in FIG. 1, which shows HPLC detection of relevant compounds produced by Lactobacillus strains in M9-medium supplemented with L-tryptophan. An overview of the tested strain numbers and the species is shown in table 3.

TABLE 3
Strain numbers and species of tested Lactobacillus strains.

| Strain No. | Species (/DSM number) |
|---|---|
| 5 | Lactobacillus paracasei (Lacticaseibacillus paracasei) subsp. |
| | paracasei |
| 13 | Lactobacillus hilgardii (Lentilactobacillus hilgardii) |
| 25 | Lactobacillus brevis (Levilactobacillus brevis) DSM 33429 |
| 56 | Lactobacillus reuteri (Limosilactobacillus reuteri) |
| 73 | Lactobacillus crispatus |
| 123 | Lactobacillus mucosae |
| 149 | Lactobacillus delbrueckii subsp. delbrueckii DSM 33431 |
| 176 | Lactobacillus crispatus |
| 207 | Lactobacillus gasseri |
| 213 | Lactobacillus crispatus |
| 275 | Lactobacillus gasseri |
| 292 | Lactobacillus crispatus |
| 310 | Lactobacillus crispatus |
| 368 | Lactobacillus plantarum (Lactiplantibacillus plantarum) |
| 370 | Lactobacillus plantarum subsp. argentoratensis DSM 33447 |
| 426 | Lactobacillus plantarum (Lactiplantibacillus plantarum) |
| 432 | Lactobacillus sakei |
| 460 | Lactobacillus paracasei (Lacticaseibacillus paracasei) subsp. |
| | paracasei |
| 474 | Lactobacillus delbrueckii subsp. lactis |
| 486 | Lactobacillus plantarum subsp. argentoratensis DSM 33449 |

As can be seen in FIG. 1 the most abundant compounds produced after incubation of Lactobacillus strains in medium containing L-tryptophan are indole-3-lactic acid, indole-3-acetic acid and kynurenine. Surprisingly some strains are able to produce these compounds in very high concentrations.

We observed that the average production of indole-3 lactic acid was higher within the Lactobacillus plantarum species as compared to others. When comparing a large number of strains of the species Lactobacillus plantarum, we observed that the strains DSM 33449 and DSM 33447 produced surprisingly high amounts of indole-3 lactic acid, exceeding all other tested strains by on average more than 200% and the next best alternative strain by 39% and 27%, respectively (see table 4).

TABLE 4
Production of indole-3 lactic acid by different Lactobacillus plantarum
species. AUC (area under the curve) values were retrieved from HPLC
analyses and correspond to metabolite concentration levels.

| | | Indole-3 |
|---|---|---|
| | | lactic |
| Strain ID | Species | acid AUC |
| 486 (DSM | Lactobacillus plantarum subsp. argentoratensis | 458204 |
| 33449) | | |
| 370 (DSM | Lactobacillus plantarum subsp. argentoratensis | 421848 |
| 33447) | | |
| 526 | Lactobacillus plantarum subsp. argentoratensis | 329074 |
| 448 | Lactobacillus plantarum | 318523 |
| 372 | Lactobacillus plantarum subsp. argentoratensis | 304690 |
| 461 | Lactobacillus plantarum subsp. argentoratensis | 302820 |
| 443 | Lactobacillus plantarum subsp. argentoratensis | 293886 |
| 399 | Lactobacillus plantarum subsp. argentoratensis | 291181 |
| 368 | Lactobacillus plantarum | 287553 |
| 462 | Lactobacillus plantarum subsp. argentoratensis | 276991 |
| 380 | Lactobacillus plantarum | 273773 |
| 375 | Lactobacillus plantarum subsp. argentoratensis | 272461 |
| 393 | Lactobacillus plantarum subsp. argentoratensis | 269783 |
| 436 | Lactobacillus plantarum subsp. argentoratensis | 262361 |
| 379 | Lactobacillus plantarum subsp. argentoratensis | 253945 |
| 381 | Lactobacillus plantarum subsp. argentoratensis | 251116 |
| 367 | Lactobacillus plantarum | 246517 |
| 388 | Lactobacillus plantarum subsp. argentoratensis | 244058 |
| 397 | Lactobacillus plantarum | 243193 |
| 559 | Lactobacillus plantarum | 242593 |
| 384 | Lactobacillus plantarum subsp. argentoratensis | 241394 |
| 596 | Lactobacillus plantarum | 239864 |
| 383 | Lactobacillus plantarum subsp. argentoratensis | 239018 |
| 389 | Lactobacillus plantarum subsp. argentoratensis | 234042 |
| 392 | Lactobacillus plantarum | 234002 |
| 523 | Lactobacillus plantarum subsp. argentoratensis | 231168 |
| 398 | Lactobacillus plantarum | 220151 |
| 449 | Lactobacillus plantarum | 216963 |
| 390 | Lactobacillus plantarum subsp. argentoratensis | 204907 |
| 476 | Lactobacillus plantarum subsp. plantarum | 200534 |
| 501 | Lactobacillus plantarum subsp. argentoratensis | 197070 |
| 427 | Lactobacillus plantarum | 191801 |
| 522 | Lactobacillus plantarum subsp. argentoratensis | 184613 |
| 500 | Lactobacillus plantarum subsp. argentoratensis | 172310 |
| 451 | Lactobacillus plantarum | 164472 |
| 439 | Lactobacillus plantarum subsp. argentoratensis | 163409 |
| 396 | Lactobacillus plantarum subsp. argentoratensis | 154100 |
| 599 | Lactobacillus plantarum subsp. argentoratensis | 152450 |
| 425 | Lactobacillus plantarum subsp. argentoratensis | 150798 |
| 525 | Lactobacillus plantarum subsp. plantarum | 150469 |
| 386 | Lactobacillus plantarum subsp. argentoratensis | 148037 |
| 515 | Lactobacillus plantarum subsp. argentoratensis | 147878 |
| 394 | Lactobacillus plantarum subsp. argentoratensis | 144762 |
| 435 | Lactobacillus plantarum subsp. argentoratensis | 141214 |
| 595 | Lactobacillus plantarum | 138826 |
| 512 | Lactobacillus plantarum subsp. argentoratensis | 138094 |
| 452 | Lactobacillus plantarum subsp. argentoratensis | 137826 |
| 130 | Lactobacillus plantarum subsp. argentoratensis | 129477 |
| 402 | Lactobacillus plantarum subsp. argentoratensis | 127980 |
| 481 | Lactobacillus plantarum subsp. argentoratensis | 125901 |
| 391 | Lactobacillus plantarum subsp. argentoratensis | 117277 |
| 373 | Lactobacillus plantarum subsp. argentoratensis | 109611 |
| 557 | Lactobacillus plantarum subsp. plantarum | 105127 |
| 566 | Lactobacillus plantarum | 91244 |
| 387 | Lactobacillus plantarum subsp. argentoratensis | 84728 |
| 598 | Lactobacillus plantarum | 81660 |
| 459 | Lactobacillus plantarum | 79831 |
| 468 | Lactobacillus plantarum subsp. plantarum | 79513 |
| 442 | Lactobacillus plantarum | 74401 |
| 198 | Lactobacillus plantarum | 70305 |
| 66 | Lactobacillus plantarum subsp. argentoratensis | 64980 |
| 64 | Lactobacillus plantarum subsp. argentoratensis | 52640 |
| 57 | Lactobacillus plantarum | 50811 |
| 592 | Lactobacillus plantarum | 47892 |
| 510 | Lactobacillus plantarum subsp. argentoratensis | 45221 |
| 231 | Lactobacillus plantarum | 44749 |
| 273 | Lactobacillus plantarum | 43923 |
| 594 | Lactobacillus plantarum subsp. argentoratensis | 43632 |
| 454 | Lactobacillus plantarum | 40739 |
| 60 | Lactobacillus plantarum subsp. argentoratensis | 37833 |
| 63 | Lactobacillus plantarum subsp. argentoratensis | 37639 |
| 473 | Lactobacillus plantarum | 29478 |
| 35 | Lactobacillus plantarum | 27293 |
| 403 | Lactobacillus plantarum subsp. argentoratensis | 27143 |
| 199 | Lactobacillus plantarum subsp. argentoratensis | 16948 |
| 52 | Lactobacillus plantarum subsp. argentoratensis | 11538 |
| 395 | Lactobacillus plantarum subsp. argentoratensis | 11372 |
| 496 | Lactobacillus plantarum | 8815 |
| 21 | Lactobacillus plantarum subsp. argentoratensis | 8428 |
| 45 | Lactobacillus plantarum subsp. argentoratensis | 8272 |
| 4 | Lactobacillus plantarum subsp. argentoratensis | 5728 |
| 100 | Lactobacillus plantarum | 5089 |
| 62 | Lactobacillus plantarum subsp. argentoratensis | 4965 |
| 33 | Lactobacillus plantarum subsp. argentoratensis | 4920 |
| 53 | Lactobacillus plantarum subsp. argentoratensis | 4503 |
| 65 | Lactobacillus plantarum subsp. argentoratensis | 3153 |
| 74 | Lactobacillus plantarum | 1404 |
| 61 | Lactobacillus plantarum | 1087 |
| 31 | Lactobacillus plantarum subsp. plantarum | 519 |
| 382 | Lactobacillus plantarum | 128 |
| 491 | Lactobacillus plantarum subsp. argentoratensis | 79 |
| 477 | Lactobacillus plantarum | |

Likely, indole-3 acetic acid was prominent for Lactobacillus delbrueckii, and we discovered that the strain Lactobacillus delbrueckii DSM 33431 stands out against other strains of this species by exceeding the average production of this metabolite by more than 160%, and the next best alternative by 27% (see table 5).

TABLE 5
Production of indole-3 lactic acid by different Lactobacillus delbrueckii
species. AUC (area under the curve) values were retrieved from HPLC
analyses and correspond to metabolite concentration levels.

| | | Indole-3 |
|---|---|---|
| | | acetic |
| Strain ID | Species | acid AUC |
| 149 (DSM | Lactobacillus delbrueckii subsp. delbrueckii | 38205 |
| 33431) | | |
| 87 | Lactobacillus delbrueckii subsp. lactis | 30407 |
| 533 | Lactobacillus delbrueckii subsp. delbrueckii | 27801 |
| 86 | Lactobacillus delbrueckii subsp. lactis | 25952 |
| 576 | Lactobacillus delbrueckii subsp. bulgaricus | 24581 |
| 15 | Lactobacillus delbrueckii subsp. delbrueckii | 22743 |
| 43 | Lactobacillus delbrueckii subsp. delbrueckii | 5016 |
| 446 | Lactobacillus delbrueckii subsp. lactis | 3681 |
| 151 | Lactobacillus delbrueckii subsp. delbrueckii | 3464 |
| 2 | Lactobacillus delbrueckii subsp. lactis | 3442 |
| 600 | Lactobacillus delbrueckii subsp. lactis | 1129 |
| 255 | Lactobacillus delbrueckii subsp. lactis | 240 |
| 254 | Lactobacillus delbrueckii subsp. lactis | 203 |

Finally, we observed L-kynurenine production by Lactobacillus brevis, which was highest in Lactobacillus brevis DSM 33429, exceeding the species' average by 320% and the next best alternative strain by 44% (see table 6).

TABLE 6
Production of indole-3 lactic acid by different Lactobacillus brevis
species. AUC (area under the curve) values were retrieved from HPLC
analyses and correspond to metabolite concentration levels.

| | | L-Kynurenine |
|---|---|---|
| Strain ID | Species | AUC |
| 25 (DSM 33429) | Lactobacillus brevis | 228139 |
| 58 | Lactobacillus brevis | 158164 |
| 67 | Lactobacillus brevis | 149811 |
| 49 | Lactobacillus brevis | 147071 |
| 99 | Lactobacillus brevis | 42271 |
| 581 | Lactobacillus brevis | 15296 |
| 565 | Lactobacillus brevis | 12870 |
| 580 | Lactobacillus brevis | 11989 |
| 567 | Lactobacillus brevis | 10410 |
| 470 | Lactobacillus brevis | 4986 |
| 469 | Lactobacillus brevis | 4601 |
| 480 | Lactobacillus brevis | 4471 |
| 428 | Lactobacillus brevis | 4411 |
| 472 | Lactobacillus brevis | 3986 |
| 511 | Lactobacillus brevis | 2276 |

### Example 3: Lactobacillus Strains According to the Present Invention are Able to Secrete Surprisingly High Levels of ILA, IAA, and L-Kynurenine

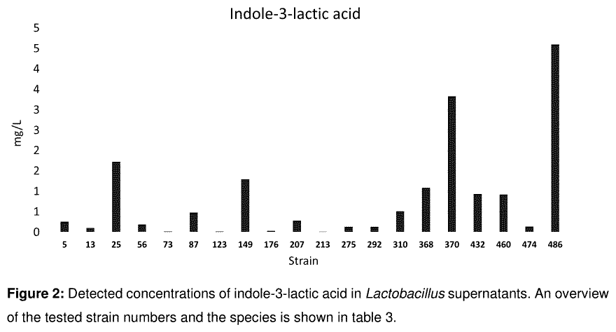

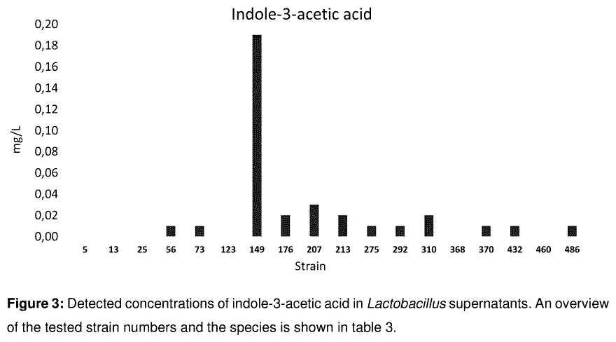

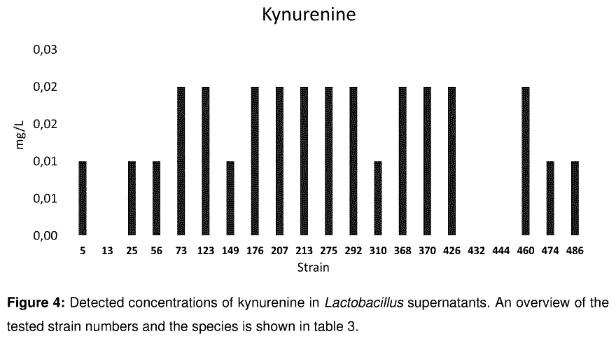

In order to determine the concentrations of relevant compounds from L-tryptophan, Lactobacillus strains were individually cultivated under anaerobic conditions in microtiter plates in MRS medium for 48 h at 37° C. Afterwards the cells were harvested by centrifugation at 4000×g for 10 min and washed with PBS buffer. Subsequently the cells were resuspended in M9-medium supplemented with 0.8 mM L-tryptophan and transferred to deep-well plates. After 6 h incubation under anaerobic conditions at 37° C., the cells were removed by centrifugation at 4000×g for 10 min and the product formation was determined by LCMSMS analysis of the supernatant. The detected concentrations of indole-3-lactic acid, indole-3-acetic acid and kynurenine in Lactobacillus supernatants are shown in FIGS. 2, 3 and 4, respectively.

The results in FIGS. 2 to 4 exhibit that certain Lactobacillus strains are able to produce one or more of the relevant products indole-3-lactic acid, indole-3-acetic acid and kynurenine at surprisingly high concentrations.

### Example 4: Secretion of Trp Metabolites by Strains from Trp is Dose-Dependent

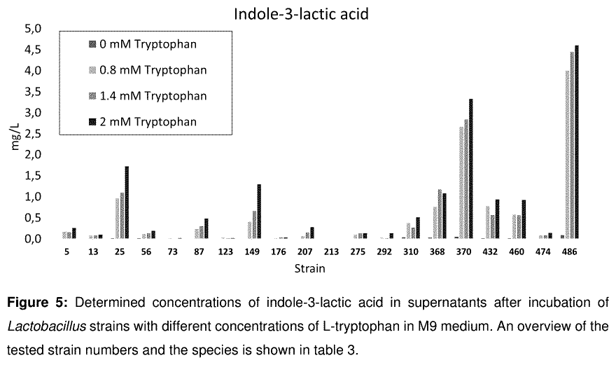

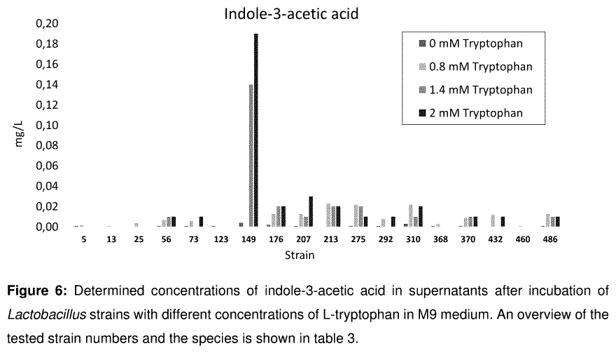

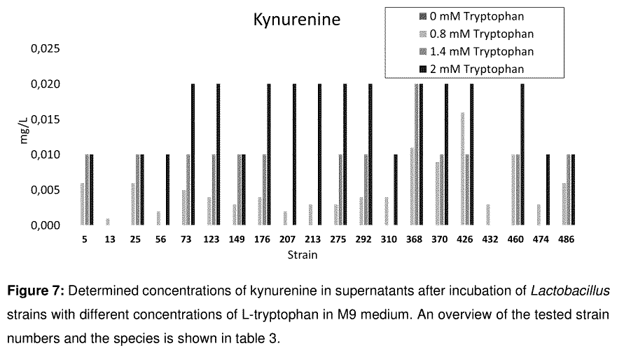

Strains were incubated in M9-medium supplemented with 0, 0.8, 1.6 or 2 mM L-tryptophan under anaerobic conditions at 37° C. After 6 h of incubation cell-free supernatants were collected and relevant compounds in supernatants were determined by LCMSMS analysis. The results are displayed in FIGS. 5 to 7. The determined concentrations of indole-3-lactic acid, indole-3-acetic acid and kynurenine in supernatants after incubation of Lactobacillus strains with different concentrations of L-tryptophan in M9 medium are shown in FIGS. 5, 6 and 7, respectively.

The results in FIGS. 5 to 7 demonstrate that the production of relevant compounds correlates with substrate concentration of L-tryptophan. In most cases the highest concentrations of products is detected when 2 mM of L-tryptophan are applied.

### Example 5: Kinetics of Secretion of Trp Metabolites by Strains from Trp

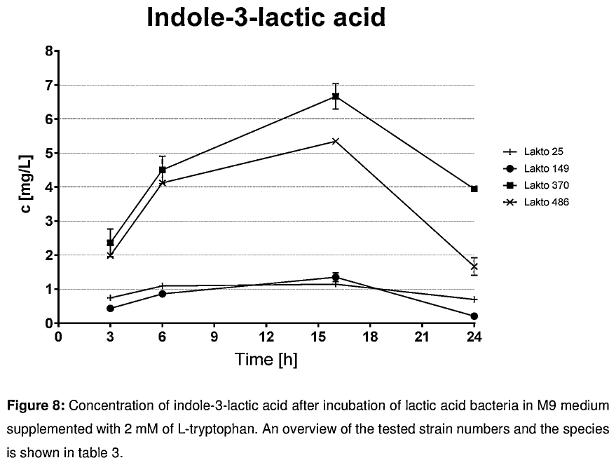

For the determination of the time-dependent product formation the strains were incubated in M9-medium supplemented with 2 mM of L-tryptophan. After 3, 6, 16 and 24 h samples were collected and analyzed by LCMSMS. The results are summarized in FIGS. 8 to 10 for the different compounds.

The data in FIG. 8 show that for all strains the highest concentration of indole-3-lactic acid is observed after 16 h of incubation. The highest concentration of indole-3-lactic acid is 6.7 mg/L for strains no. 370. Interestingly, the concentration of indole-3-lactic acid declines after 16 h for most strains which suggests that the product is consumed by the cells due to lack of nutrients after 16 h. Furthermore, it is noteworthy that indole-3-lactic acid can already be detected after 3 h for most of the strains, which is beneficial for a prospective in vivo activity.

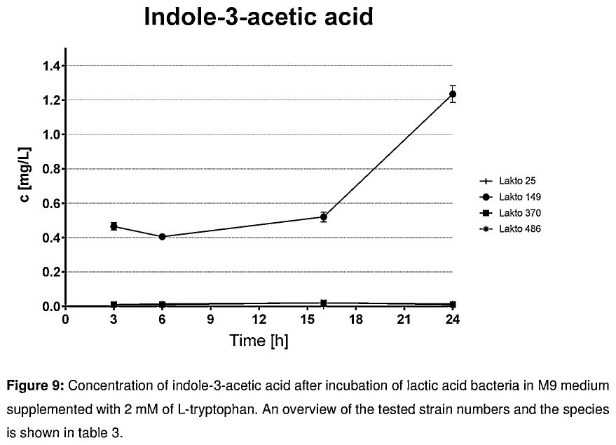

As can be seen in FIG. 9 the highest detected concentration of indole-3-acetic is observed for strain no. 149 which produces up to 1.2 mg/L after 24 h of incubation.

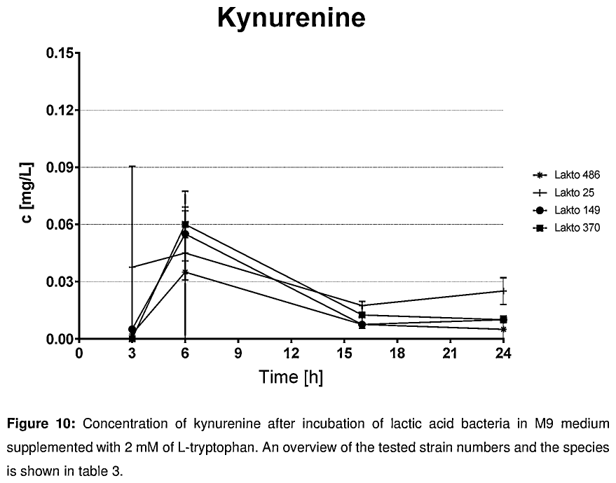

As shown in FIG. 10, the production of kynurenine is strain dependent. The highest concentration is observed after 6 h for all strains. The highest value is obtained by strain no. 370 (0.06 mg/L). For most strains, the concentration of kynurenine declines after 6 h.

### Example 6: Composition of Synbiotic Capsules Comprising a Source of L-Tryptophan and Lactobacillus Strain(s) as Food Supplement or as Drug

The following components were filled in HPMC capsules (size 00 or other).

TABLE 7
Preparations for filling into HPMC capsules.

| Compound | Capsule I | Capsule II | Capsule III |
|---|---|---|---|
| L-tryptophan* | 250 mg | 50 mg | 800 mg |
| Lactobacillus strain^{#} | 1 × 10⁷ CFU- | 1 × 10⁷ CFU- | 1 × 10⁷ CFU- |
| | 1 × 10¹¹ CFU | 1 × 10¹¹ CFU | 1 × 10¹¹ CFU |

*L-tryptophan may be added as free amino acid or modification thereof or contained in peptides or proteins.
^{#}Strain selected from Lactobacillus plantarum (Lactiplantibacillus plantarum) DSM 33447, Lactobacillus plantarum (Lactiplantibacillus plantarum) DSM 33449, Lactobacillus brevis (Levilactobacillus brevis) DSM 33429, or Lactobacillus delbrueckii DSM 33431.

The capsules may further contain amino acids selected from L-ornithine, L-aspartate, L-lysine and L-arginine.

The capsules may further contain further carbohydrate ingredients, selected from arabinoxylans, barley grain fibre, oat grain fibre, rye fibre, wheat bran fibre, inulins, fructooligosaccharides (FOS), galactooligosaccharides (GOS), resistant starch, beta-glucans, glucomannans, galactoglucomannans, guar gum and xylooligosaccharides.

The capsules may further contain one or more plant extracts, selected from ginger, cinnamon, grapefruit, parsley, turmeric, curcuma, olive fruit, panax ginseng, horseradish, garlic, broccoli, spirulina, pomegranate, cauliflower, kale, cilantro, green tea, onions, and milk thistle.

The capsules may further contain astaxanthin, charcoal, chitosan, glutathione, monacolin K, plant sterols, plant stanols, sulforaphane, collagen, hyalurone, phosphatidylcholine.

The capsules may comprise further vitamins selected from biotin, vitamin A, vitamin B1 (thiamine), vitamin B2 (riboflavin), vitamin B3 (niacin), vitamin B5 (pantothenic acid), vitamin B9 (folic acid or folate), vitamin C (ascorbic acid), vitamin D (calciferols), vitamin E (tocopherols and tocotrienols) and vitamin K (quinones) or minerals selected from sulfur, iron, chlorine, calcium, chromium, cobalt, copper, magnesium, manganese, molybdenum, iodine, selenium, and zinc.

### Example 7: Capsules Coated with Eudreguard® Biotic

HPMC capsules (size 3) were filled with a composition as described in table 7. The total capsule weight was 200 mg. The capsules were coated with an enteric coating composition as shown in table 8.

TABLE 8
Coating composition

| | | Content | | Content |
|---|---|---|---|---|
| | Dry | based on | Weight | based on |
| | substance | coating | gain | capsule |
| Compound | [g] | [%] | [%] | [%] |
| EUDRAGUARD ® | 40.8 | 36.9 | 8.2 | 6.7 |
| biotic | | | | |
| HPMC | 43.1 | 39.0 | 8.6 | 7.1 |
| Talc | 20.4 | 18.4 | 4.0 | 3.3 |
| Polyethylene | 4.3 | 3.9 | 0.9 | 0.7 |
| glycol | | | | |
| Triethyl citrate | 2.0 | 1.8 | 0.4 | 0.3 |

### REFERENCES

1. Gibson G R, Hutkins R, Sanders M E, Prescott S L, Reimer R A, Salminen S J, Scott K, Stanton C, Swanson K S, Cani P D et al: Expert consensus document: The International Scientific Association for Probiotics and Prebiotics (ISAPP) consensus statement on the definition and scope of prebiotics. Nat Rev Gastroenterol Hepatol 2017, 14(8):491-502.
2. Hill C, Guarner F, Reid G, Gibson G R, Merenstein D J, Pot B, Morelli L, Canani R B, Flint H J, Salminen S et al: Expert consensus document. The International Scientific Association for Probiotics and Prebiotics consensus statement on the scope and appropriate use of the term probiotic. Nat Rev Gastroenterol Hepatol 2014, 11(8):506-514.
3. Pandey K R, Naik S R, Vakil B V: Probiotics, prebiotics and synbiotics—a review. J Food Sci Technol 2015, 52(12):7577-7587.
4. Dodd D, Spitzer M H, Van Treuren W, Merrill B D, Hryckowian A J, Higginbottom S K, Le A, Cowan T M, Nolan G P, Fischbach M A et al: A gut bacterial pathway metabolizes aromatic amino acids into nine circulating metabolites. Nature 2017, 551(7682):648-652.
5. Lamas B, Richard M L, Leducq V, Pham H P, Michel M L, Da Costa G, Bridonneau C, Jegou S, Hoffmann T W, Natividad J M et al: CARD9 impacts colitis by altering gut microbiota metabolism of tryptophan into aryl hydrocarbon receptor ligands. Nat Med 2016, 22(6):598-605.
6. Ogyu K, Kubo K, Noda Y, Iwata Y, Tsugawa S, Omura Y, Wada M, Tarumi R, Plitman E, Moriguchi S et al: Kynurenine pathway in depression: A systematic review and meta-analysis. Neurosci Biobehav Rev 2018, 90:16-25.
7. Osadchiy V, Labus J S, Gupta A, Jacobs J, Ashe-McNalley C, Hsiao E Y, Mayer E A: Correlation of tryptophan metabolites with connectivity of extended central reward network in healthy subjects. PLOS One 2018, 13(8):e0201772.
8. Qi H, Li Y, Yun H, Zhang T, Huang Y, Zhou J, Yan H, Wei J, Liu Y, Zhang Z et al: Lactobacillus maintains healthy gut mucosa by producing L-Ornithine. Commun Biol 2019, 2:171.
9. Cervantes-Barragan L, Chai J N, Tianero M D, Di Luccia B, Ahern P P, Merriman J, Cortez V S, Caparon M G, Donia M S, Gilfillan S et al: Lactobacillus reuteri (Limosilactobacillus reuteri) induces gut intraepithelial CD4(+)CD8alphaalpha(+) T cells. Science 2017, 357(6353):806-810.
10. Cheng Y, Jin U H, Allred C D, Jayaraman A, Chapkin R S, Safe S: Aryl Hydrocarbon Receptor Activity of Tryptophan Metabolites in Young Adult Mouse Colonocytes. Drug Metab Dispos 2015, 43(10):1536-1543.
11. Zelante T, lannitti R G, Cunha C, De Luca A, Giovannini G, Pieraccini G, Zecchi R, D'Angelo C, Massi-Benedetti C, Fallarino F et al: Tryptophan catabolites from microbiota engage aryl hydrocarbon receptor and balance mucosal reactivity via interleukin-22. Immunity 2013, 39(2):372-385.
12. Krishnan S, Ding Y, Saedi N, Choi M, Sridharan G V, Sherr D H, Yarmush M L, Alaniz R C, Jayaraman A, Lee K: Gut Microbiota-Derived Tryptophan Metabolites Modulate Inflammatory Response in Hepatocytes and Macrophages. Cell Rep 2018, 23(4): 1099-1111.
13. Bock K W: Human and rodent aryl hydrocarbon receptor (AHR): from mediator of dioxin toxicity to physiologic AHR functions and therapeutic options. Biol Chem 2017, 398(4):455-464.
14. Venkatesh M, Mukherjee S, Wang H, Li H, Sun K, Benechet A P, Qiu Z, Maher L, Redinbo M R, Phillips R S et al: Symbiotic bacterial metabolites regulate gastrointestinal barrier function via the xenobiotic sensor PXR and Toll-like receptor 4. Immunity 2014, 41(2):296-310.
15. Wilck N, Matus M G, Kearney S M, Olesen S W, Forslund K, Bartolomaeus H, Haase S, Mahler A, Balogh A, Marko L et al: Salt-responsive gut commensal modulates TH17 axis and disease. Nature 2017, 551(7682):585-589.

### NUCLEOTIDE SEQUENCES

SEQ ID NO:1 Lactobacillus plantarum strain DSM 33447 groL
SEQ ID NO: 2 Lactobacillus plantarum strain DSM 33447 gyrB
SEQ ID NO: 3 Lactobacillus plantarum strain DSM 33447 dnaA
SEQ ID NO: 4 Lactobacillus plantarum strain DSM 33447 rpsK
SEQ ID NO: 5 Lactobacillus plantarum strain DSM 33447 rpmB
SEQ ID NO: 6 Lactobacillus plantarum strain DSM 33447 consensus 16 rDNA
SEQ ID NO: 7 Lactobacillus delbrueckii strain DSM 33431 groL
SEQ ID NO: 8 Lactobacillus delbrueckii strain DSM 33431 gyrB
SEQ ID NO: 9 Lactobacillus delbrueckii strain DSM 33431 dnaA
SEQ ID NO: 10 Lactobacillus delbrueckii strain DSM 33431 rpsK
SEQ ID NO: 11 Lactobacillus delbrueckii strain DSM 33431 rpmB
SEQ ID NO: 12 Lactobacillus delbrueckii strain DSM 33431 16 rDNA
SEQ ID NO: 13 Lactobacillus brevis strain DSM 33429 groL
SEQ ID NO: 14 Lactobacillus brevis strain DSM 33429 gyrB
SEQ ID NO: 15 Lactobacillus brevis strain DSM 33429 dnaA
SEQ ID NO: 16 Lactobacillus brevis strain DSM 33429 rpsK
SEQ ID NO: 17 Lactobacillus brevis strain DSM 33429 rpmB
SEQ ID NO: 18 Lactobacillus brevis strain DSM 33429 consensus 16 rDNA
SEQ ID NO: 19 Lactobacillus plantarum subspecies argentoratensis DSM 33449 groL
SEQ ID NO: 20 Lactobacillus plantarum subspecies argentoratensis DSM 33449 gyrB
SEQ ID NO: 21 Lactobacillus plantarum subspecies argentoratensis DSM 33449 dnaA
SEQ ID NO: 22 Lactobacillus plantarum subspecies argentoratensis DSM 33449 rpsK
SEQ ID NO: 23 Lactobacillus plantarum subspecies argentoratensis DSM 33449 rpmB
SEQ ID NO: 24 Lactobacillus plantarum subspecies argentoratensis DSM 33449 consensuses 16 rDNA

1-17. (canceled)

18. A preparation comprising:
at least one probiotic strain belonging to the genus Lactobacillus paracasei (Lacticaseibacillus paracasei): Lactobacillus brevis (Levilactobacillus brevis): Lactobacillus delbrueckii: Lactobacillus crispatus: Lactobacillus plantarum (Lactiplantibacillus plantarum): Lactobacillus plantarum subspecies argentoratensis: Lactobacillus reuteri (Limosilactobacillus reuteri): or Lactobacillus hilgardii (Lentilactobacillus hilgardii); and
L-tryptophan or a dipeptide containing L-tryptophan: or
a foodstuff, fruit or plant or meat extract containing L-tryptophan.

19. The preparation of claim 18, wherein the preparation comprises the probiotic strain and a foodstuff, fruit plant or meat extract containing L-tryptophan at a concentration of at least 0.01 weight-%.

20. The preparation of claim 18, wherein the preparation comprises the probiotic strain and a foodstuff, fruit plant or meat extract containing L-tryptophan at a concentration of at least 0.10 weight-%; and the foodstuff, fruit, plant or meat extract is selected from the group consisting of: soy beans: cashew nuts: peanuts: lentils: oat; quark: egg: tuna; and chicken.

21. The preparation of claim 18, further comprising a targeted-release formulation for delayed release or enteric or colonic release.

22. The preparation of claim 21, wherein the targeted-release formulation comprises a coating comprising at least one pH dependent polymer or biodegradable polymer, selected from the group consisting of: methyl acrylate-methacrylic acid copolymers: cellulose acetate phthalate (CAP): cellulose acetate succinate: hydroxypropyl methyl cellulose phthalate; hydroxypropyl methyl cellulose acetate succinate (hypromellose acetate succinate); polyvinyl acetate phthalate (PVAP): methyl methacrylate-methacrylic acid copolymers; shellac; cellulose acetate trimellitate; sodium alginate; and zein.

23. The preparation of claim 22, wherein the coating comprises a polymer polymerized from 10 to 30% by weight methyl methacrylate, 50 to 70% by weight methyl acrylate and 5 to 15% by weight methacrylic acid.

24. The preparation of claim 23, wherein the coating comprises 15 to 50% by weight of a polymer polymerized from 20 to 30% by weight methyl methacrylate, 60 to 70% by weight methyl acrylate and 8 to 12% by weight methacrylic acid.

25. The preparation of claim 18, wherein the probiotic strains are selected from the group consisting of: Lactobacillus brevis (Levilactobacillus brevis) DSM 33429; Lactobacillus delbrueckii DSM 33431: Lactobacillus plantarum (Lactiplantibacillus plantarum) DSM 33447; and Lactobacillus plantarum (Lactiplantibacillus plantarum) DSM 33449.

26. The preparation of claim 18, wherein the probiotic strain is Lactobacillus plantarum (Lactiplantibacillus plantarum) and exhibits the following characteristics:
a) a groL sequence with a sequence identity of at least 95% to the polynucleotide sequence of SEQ ID NO: 1;
b) a gyrB sequence with a sequence identity of at least 95% to the polynucleotide sequence of SEQ ID NO: 2;
c) a dnaA sequence with a sequence identity of at least 95% to the polynucleotide sequence of SEQ ID NO: 3;
d) a rpsK sequence with a sequence identity of at least 95% to the polynucleotide sequence of SEQ ID NO: 4;
e) a rpmB sequence with a sequence identity of at least 95% to the polynucleotide sequence of SEQ ID NO: 5;
f) a consensus 16 rDNA sequence with a sequence identity of at least 95% to the polynucleotide sequence of SEQ ID NO: 6.

27. The preparation of claim 18, wherein the probiotic strain is Lactobacillus plantarum (Lactiplantibacillus plantarum) and exhibits the following characteristics:
a) a groL sequence with a sequence identity of at least 99.5% to the polynucleotide sequence of SEQ ID NO: 1;
b) a gyrB sequence with a sequence identity of at least 99.5% to the polynucleotide sequence of SEQ ID NO: 2;
c) a dnaA sequence with a sequence identity of at least 99.5% to the polynucleotide sequence of SEQ ID NO: 3;
d) a rpsK sequence with a sequence identity of at least 99.5% to the polynucleotide sequence of SEQ ID NO: 4;
e) a rpmB sequence with a sequence identity of at least 99.5% to the polynucleotide sequence of SEQ ID NO: 5;
f) a consensus 16 rDNA sequence with a sequence identity of at least 99.5%, to the polynucleotide sequence of SEQ ID NO: 6.

28. The preparation of claim 18, wherein the probiotic strain is Lactobacillus delbrueckii and exhibits the following characteristics:
a) a groL sequence with a sequence identity of at least 95% to the polynucleotide sequence of SEQ ID NO: 7;
b) a gyrB sequence with a sequence identity of at least 95% to the polynucleotide sequence of SEQ ID NO: 8;
c) a dnaA sequence with a sequence identity of at least 95% to the polynucleotide sequence of SEQ ID NO: 9;
d) a rpsK sequence with a sequence identity of at least 95% to the polynucleotide sequence of SEQ ID NO: 10;
e) a rpmB sequence with a sequence identity of at least 95% to the polynucleotide sequence of SEQ ID NO: 11;
f) a consensus 16 rDNA sequence with a sequence identity of at least 95% to the polynucleotide sequence of SEQ ID NO: 12.

29. The preparation of claim 18, wherein the probiotic strain is Lactobacillus delbrueckii and exhibits the following characteristics:
a) a groL sequence with a sequence identity of at least 99.5% to the polynucleotide sequence of SEQ ID NO: 7;
b) a gyrB sequence with a sequence identity of at least 99.5% to the polynucleotide sequence of SEQ ID NO: 8;
c) a dnaA sequence with a sequence identity of at least 99.5% to the polynucleotide sequence of SEQ ID NO: 9;
d) a rpsK sequence with a sequence identity of at least 99.5% to the polynucleotide sequence of SEQ ID NO: 10;
e) a rpmB sequence with a sequence identity of at least 99.5% to the polynucleotide sequence of SEQ ID NO: 11;
f) a consensus 16 rDNA sequence with a sequence identity of at least 99.5% to the polynucleotide sequence of SEQ ID NO: 12.

30. The preparation of claim 18, wherein the probiotic strain is Lactobacillus brevis (Levilactobacillus brevis) and exhibits the following characteristics:
a) a groL sequence with a sequence identity of at least 95% to the polynucleotide sequence of SEQ ID NO: 13;
b) a gyrB sequence with a sequence identity of at least 95% to the polynucleotide sequence of SEQ ID NO: 14;
c) a dnaA sequence with a sequence identity of at least 95% to the polynucleotide sequence of SEQ ID NO: 15;
d) a rpsK sequence with a sequence identity of at least 95% to the polynucleotide sequence of SEQ ID NO: 16;
e) a rpmB sequence with a sequence identity of at least 95% to the polynucleotide sequence of SEQ ID NO: 17;
f) a consensus 16 rDNA sequence with a sequence identity of at least 95% to the polynucleotide sequence of SEQ ID NO: 18.

31. The preparation of claim 18, wherein the probiotic strain is Lactobacillus brevis (Levilactobacillus brevis) and exhibits the following characteristics:
a) a groL sequence with a sequence identity of at least 99.5% to the polynucleotide sequence of SEQ ID NO: 13;
b) a gyrB sequence with a sequence identity of at least 99.5% to the polynucleotide sequence of SEQ ID NO: 14;
c) a dnaA sequence with a sequence identity of at least 99.5% to the polynucleotide sequence of SEQ ID NO: 15;
d) a rpsK sequence with a sequence identity of at least 99.5% to the polynucleotide sequence of SEQ ID NO: 16;
e) a rpmB sequence with a sequence identity of at least 99.5% to the polynucleotide sequence of SEQ ID NO: 17;
f) a consensus 16 rDNA sequence with a sequence identity of at least 99.5% to the polynucleotide of SEQ ID NO: 18.

32. The preparation of claim 18, wherein the probiotic strain is Lactobacillus plantarum subspecies argentoratensis and exhibits the following characteristics:
a) a groL sequence with a sequence identity of at least 95% to the polynucleotide sequence of SEQ ID NO: 19;
b) a gyrB sequence with a sequence identity of at least 95% to the polynucleotide sequence of SEQ ID NO: 20;
c) a dnaA sequence with a sequence identity of at least 95% to the polynucleotide sequence of SEQ ID NO: 21;
d) a rpsK sequence with a sequence identity of at least 95% to the polynucleotide sequence of SEQ ID NO: 22;
e) a rpmB sequence with a sequence identity of at least 95% to the polynucleotide sequence of SEQ ID NO: 23;
f) a consensus 16 rDNA sequence with a sequence identity of at least 95% to the polynucleotide sequence of SEQ ID NO: 24.

33. The preparation of claim 18, wherein the probiotic strain is Lactobacillus plantarum subspecies argentoratensis and exhibits the following characteristics:
a) a groL sequence with a sequence identity of at least 99.5% to the polynucleotide sequence of SEQ ID NO: 19;
b) a gyrB sequence with a sequence identity of at least 99.5% to the polynucleotide sequence of SEQ ID NO: 20;
c) a dnaA sequence with a sequence identity of at least 99.5% to the polynucleotide sequence of SEQ ID NO: 21;
d) a rpsK sequence with a sequence identity of at least 99.5% to the polynucleotide sequence of SEQ ID NO: 22;
e) a rpmB sequence with a sequence identity of at least 99.5% to the polynucleotide sequence of SEQ ID NO: 23;
f) a consensus 16 rDNA sequence with a sequence identity of at least 99.5% to the polynucleotide sequence of SEQ ID NO: 24.

34. The preparation of claim 18, wherein the probiotic strain is present in a dose range of 1×10⁷-1×10¹¹ colony-forming units (CFU).

35. The preparation of claim 18, wherein L-tryptophan is present in an amount of at least 10 mg.

36. A food supplement or food product, comprising the preparation of claim 18, and at least one further food ingredient.

37. The food supplement or food product of claim 36, wherein the further food ingredient is selected from the group consisting of: amino acids; proteins; carbohydrates; fats; further probiotics; prebiotics; enzymes; vitamins; immune modulators; milk replacers; minerals; coccidiostats; acid-based products; medicines; and combinations thereof.


SEQUENCE LISTING (24 sequences)

SEQ ID NO: 1 - DNA, 1626 bp, Lactobacillus plantarum strain DSM 33447
atggctaaagaattaaagttctctgaagatgcacgttcagcgatgctaaaaggtgtcgatcaattagctgacacagttaagtcaacgttaggtcctaagggtcgcaacgttgttttggaacaatcatatggttcaccaacaattactaatgatggtgtaacgattgctaaggcgatcgaattagacgatcatttcgaaaacatgggtgctaagttagtttctgaagttgcttcaaagactaatgacatcgctggtgatgggacgactactgcaacggtcttaacacaatcaatcgttaatgaaggtatgaagaacgttacggccggtgctaaccctgttggcattcgtcgtgggattgaagaagctactaagacggcggttgactcattacacgctatggcacacgaagttaagacgcaagaagatattgcgcaaatcgcttctgtatcttcagcaagtgaagaaactggtaaattgattgccgaagccatggaaaaagttggtcatgacggtgttatcacgattgaagaatcacgtggtgttgatactagtttagacgttgttgaagggatgcaattcgaccgcggctacttatcacaatacatggttactgataatgataagatggaagcggatcttgacaatccatatatcttaattactgataagaagatttcaaacattcaagatatcttaccactattacaatccatcgttgaacaaggcaagccattgttgatcattgctgatgacatttctggtgaagctttaccaaccttagtcttgaacaagatgcgtgggacgtttaacgttgtcgccgttaaggcacccggttttggtgatcggcgtaaggaacaattacaagatatcgctatcttaactggcgggacggttatcactgacgaccttggccttgaattgaaggacacgaccatcgatcaattaggtcaagccaacaaagttacggttactaaggataacaccaccattgttgaaggcgctggttccaaggatgctatctcagaacgggttgaatttatccgtaaccaaatcggtgaaacaacttctgactttgacaaagaaaagttacaagaacgtttagctaaattagctggtggggttgccgttgttcgtgtcggtgccgctactgaaactgaattgaaggaacgtaaataccggattgaagatgctttgaacgcaactcgggccgccgttgaagaaggctttgttgctggtggtggtactgctttgattaacgttatcaaagatgttgctgcattgaaggaaactggtgacgttcaaactgggatcaacattgttaaacgtgctttggaagaaccagttcgccaaatcgctgaaaatgctggtttggaaggctctgttatcgttgaaaagatgaaggaacaaaagccaggtgttggtttcaacgccgcaactgatgaatgggttgacatgatcaaagctggtatcgtggacccaactaaggtaacgcgttctgctttacaaaatgccgcttctgtttcagcccttctcttaacgactgaagccgttgtcgctgaaaaacctgaagaaaatgcaccagctgcaccagccgcaccaaacccaggtatgggcggtatgatgtaa

SEQ ID NO: 2 - DNA, 1947 bp, Lactobacillus plantarum strain DSM 33447
ttgaccgaaaacgaaaaagaaaccaaacttgaacaggctcgcgaatatgatgctagtcaaattcaagttttggaaggtttagaagctgtccggaaacgtcccggaatgtatatcggtacgacgagtagccaaggactccaccacttagtttgggaaattgttgataatggaattgatgaagccctagcagggtttgcaaacgtgatccacgtgaccgttgaaaaggataatagtattacggttactgataacggtcgtgggattcctgttgatattcaagaaaaaacgggtaaaccagcgcttgaaacggtttatacgattttacatgccggtggtaaattcggtggtggcggttataaagtctctggtggtcttcacggggtcggggcttccgttgttaacgccttatcatcgaccctggacgtcaaagtaactcggaatggtcatgtttattacatggactttgaacgtggaaaggttaaaacaccgatgaagatcattggtgatgcgcctgatgatgctcatggtacggcggttcattttgttccggatcctgatattttccgggaaacgaccacctatgacattaatattttgacgactcggattcgcgaactcgctttcttaaataagggattacggatcacgattcgggataatcgccctgatgaacccactgaagatgactttctctacgaaggtgggattcgtcactatgttgaatatctagataagaataagaccgttctatttccagaaccgatttatgtggaaggggaacagaatgggattacggtagaagttgcgttgcaatatactgacgactaccacagtaatttgatgaccttcaccaacaatattcatacgtacgaaggtgggacgcatgaagaaggtttcaaacgtgctttgacgcgggtcatcaacgactatgctcgtaagaataacttgttgaaggaaagcgatgcgaatctatctggcgaagatgttcgtgaagggatgacggctgttgtcagcgtcaagcatccggatcctcaatttgaaggtcaaacgaagacgaaacttggtaattccgatgctcgggcagcagttgaccggttattctcagaacactttagcaagtacttgatggaaaacccaagtgttgcccgtaaagtggtcgacaaagggttacttgcatccaaggcccgtgtcgctgctaagcgtgcgcgggaagttacgcgaaaaaagagtggcttggaaattagtaatttaccaggtaagttggcggacaatagtagtaaagaccctgaaattagtgagttgttcattgtcgaaggggattctgccggtgggtcggctaagcagggacgttcccggttgacgcaagcgattttaccgattcggggtaaaattttgaacgttgaaaaggccagcattgataagattttagccaatgaagaaatccggtcattgtttacggcaatgggaaccggcttcggtggtgacttcgacttgagcaaagccaattatcacaaattaattatcatgactgatgccgatgtcgatggggcccatattcggaccttgttattgacgttgttttaccggtacatgcggccattagttgacgctggctttgtgtacatcgctcaaccaccgttgtaccaagtccggcaaggtaaatttgtgaagtatattgactcagatgaagaattgagtgaggtcatggggcaattggcaccatcaccgaaaccagttgttcaacggtacaaggggcttggtgaaatggacgcggaacaattatgggaaacgaccatggatccagacaagcggcgcttattacgggttcgtgatgaagacgctgcggatgccgatggggtcttctcgatgctgatgggggaccaagtcaaaccacggcgagaatttattgaagataatgccaagttcgtccaggatctggatgtttag

SEQ ID NO: 3 - DNA, 1368 bp, Lactobacillus plantarum strain DSM 33447
gtgctggaacgtaatgacttatggaatctcattaagttctctatggaacaagatctgtccaagatcacgtttcaaacgtttgtcgaaccggcaaaaccactccaactcgataaagcccaaatgacgatcgaagtcccaacacaacttcatcgtgactattgggagaaaaacttggccgcaaagttcacggacattgcgatgcaagcgactaacgagcagattcggccggtcatgataacggaagaagaacgtcagcaacttacgagagacaaggactcgcaagtgactaccggtaacgttgcgggacaacaaccaacaaccgcaactacccccacatttatgcgggaaacgaaactcaacccgaaatacacttttgatactttcgtgatcggtaaaggcaatcaaatggcccatgccgctgcgttagttgtgtcggaagaacccggcaccatgtataatccgttgtttttctacgggggcgttggtctgggaaaaacccacctaatgcacgctatcggtaacaaattgttagaaaccgatccgactagtaacattaaatatgtgactagcgaatcttttacgaatgaattaattaatgccattcaaactaaaaaacaggaggcgttccgcgaagaatatcggaacgttgacctgttattagtcgacgacattcaattttttgccaataaggaagcaacccaagaagagttcttccatacatttaatgctttatatgaagatgataagcaaatcgtgcttacatccgatcgcttaccgaacgaaattccgcaactccaagatcgcctagtttctaggtttaagtggggattatccgttgatattaccccacctgatctcgagacgcggattgctattttgcgcaataaagccgatcttgaagggatcgaaatccctgacgatacgttaagttatatcgccggtcagatcgactcaaacgtgcgagaactcgaaggtgccttagcacgagtgcaggcatactcacgactgaataattcaccgattaccaccagtttagtggcggatgcattgaagagtttgcaccttagcagtaaactatcagaggtttcgattcccgtcattcaggaaaaagtcgccaagtacttccatgtaacaatggctgaccttaagggaaagaaacgcaataaacaaatcgtgattccacgtcaaattgccatgtatttatcacgcgaactgactgaaagttcacttccgcgcatcggtaacgagtttggtggtaaggaccatacgactgtgatccatgcgcatgataaaatcactaaggcactcaaaactgatgctgaattacaaaaagccgtgggtgatttgacaggccagctcaaatcttag

SEQ ID NO: 4 - DNA, 390 bp, Lactobacillus plantarum strain DSM 33447
atggcaactagaaagacaacccgtcgtcgtcgggtaaagaagaacattgaatctggtgtggctcacatccattcaacgttcaacaacacacttgttatgatcactgatatgcaagggaacgccattgcatggtcatcagctggttcattaggtttcaaaggtagtcgtaagtcaacaccttttgctgctcaaatggctgcagaagctgctgctaaggcatcaatggaacatggtatgaagactgttgaagtcgctgttaagggtcctggttcaggccgtgaagccgctatccgtgctttacaagctactggtttggaagttagcgcaattcgcgatgttacgccagtgcctcacaatggttctcgtcctccaaagcgtcgtcgtgtttaa

SEQ ID NO: 5 - DNA, 186 bp, Lactobacillus plantarum strain DSM 33447
atggcaaaagactacgttacaggcaaacggacgcactttggtaacacgcgttctcacgctttaaaccacagccgccgcagctggaaagctaacttgcaaaaagttcgcattttagttgatggtaaaccaaagaaggtttgggtttcagctcggactttgaaatcaggtaaagttacgcgcgtttag

SEQ ID NO: 6 - DNA, 1563 bp, Lactobacillus plantarum strain DSM 33447
tttgagagtttgatcctggctcaggacgaacgctggcggcgtgcctaatacatgcaagtcgaacgaactctggtattgattggtgcttgcatcatgatttacatttgagtgagtggcgaactggtgagtaacacgtgggaaacctgcccagaagcgggggataacacctggaaacagatgctaataccgcataacaacttggaccgcatggtccgagtttgaaagatggcttcggctatcacttttggatggtcccgcggcgtattagctagatggtggggtaacggctcaccatggcaatgatacgtagccgacctgagagggtaatcggccacattgggactgagacacggcccaaactcctacgggaggcagcagtagggaatcttccacaatggacgaaagtctgatggagcaacgccgcgtgagtgaagaagggtttcggctcgtaaaactctgttgttaaagaagaacatatctgagagtaactgttcaggtattgacggtatttaaccagaaagccacggctaactacgtgccagcagccgcggtaatacgtaggtggcaagcgttgtccggatttattgggcgtaaagcgagcgcaggcggttttttaagtctgatgtgaaagccttcggctcaaccgaagaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagtatgggtagcaaacaggattagataccctggtagtccataccgtaaacgatgaatgctaagtgttggagggtttccgcccttcagtgctgcagctaacgcattaagcattccgcctggggagtacggccgcaaggctgaaactcaaaggaattgacgggggcccgcacaagcggtggagcatgtggtttaattcgaagctacgcgaagaaccttaccaggtcttgacatactatgcaaatctaagagattagacgttcccttcggggacatggatacaggtggtgcatggttgtcgtcagctcgtgtcgtgagatgttgggttaagtcccgcaacgagcgcaacccttattatcagttgccagcattaagttgggcactctggtgagactgccggtgacaaaccggaggaaggtggggatgacgtcaaatcatcatgccccttatgacctgggctacacacgtgctacaatggatggtacaacgagttgcgaactcgcgagagtaagctaatctcttaaagccattctcagttcggattgtaggctgcaactcgcctacatgaagtcggaatcgctagtaatcgcggatcagcatgccgcggtgaatacgttcccgggccttgtacacaccgcccgtcacaccatgagagtttgtaacacccaaagtcggtggggtaaccttttaggaaccagccgcctaaggtgggacagatgattagggtgaagtcgtaacaaggtagccgtaggagaacctgcggctggatcacctcctt

SEQ ID NO: 7 - DNA, 1614 bp, Lactobacillus delbrueckii strain DSM 33431
atggcaaaagacatcaagttttcagaagaagcaagacgttcacttttaaacggggttaacaagctggccaacacggtcaagacgaccttggggcctaagggcagaaacgtagttttggaacaaagctacggcgccccaacgattaccaacgacggcgtgactatcgccaaggcaattgaactggaagaccactacgaaaacatgggggccaagctggttgctgaagctgcttcaaagaccaacgacatcgctggtgatggtacgaccacggccactgttttgacccaagccatcgtgcaagaagggatgaagaacgtggttgccggggctaacccagttggcattcgccgcgggattgaaaaggccacccaagcagccgttgaccaattgcacaagaacagccacgaagtttccagccgggaccaaattgcccaagttgcttcaatctcaagtgcttcaaaggaaatcggcgacttgatcgctgaagccatggaaaaggtcggcaaagacggtgttatcaccattgaagactcccgcgggatcgaaactgaactgagcgtggttgaagggatgcaattcgaccgcggctacctgtcccaatacatggtaacggacaacgacaagatggaagctgacttggaaaacccatacatcttgatcactgacaagaagatttccaacatccaggacattttgccaatgttgcaggaaatcgtgaaagaaggccgctcactcctaatcatcgctgatgacgtgactggcgaagctttgccaactcttgttttgaacaagatccgcggtaccttcaacgttgttgctgttaaggctcctggctttggtgaccgccgcaaggaacaactggctgacatcgctgctttgactggcggtacggtaatctctgaagacctgggcctggaactcaaggacacccagctcagccaattgggtcaagcccgccgggtaactatcaccaaggactcaactactatcgttgacggctctggcgctaaggaagccatccaagaacgggttgacaccatcagaaagcaaatcgaagacacttcttctgacttcgacaagaagaagctgcaagaacgtctggccaagctgactggcggcgtagccgtaatccacgtcggtgctgctactgaaactgaactcaaggaacgccgctaccgggtagaagacgccttgaacgctacccgtgctgctgttgaagaaggctacgtagctggtggtggtactgctttggttaacgttgaagaagctgttaaggcaatcaagggtgacactgctgacgaacaaaccgggatcaacatcgtggcccgtgccttgactgccccagttcgccaaatcgctgaaaacgccggccaagaaggctcagtcgttgttgaccacctgcgcaaggttgacccggaagtcggctacaacgctgctgaagacaagtatgtcaacatgattgacgaagggatcatcgacccaaccaaggtgacccggtcagccctgcaaaatgccgcttcaatcgccggcctcttgctgaccactgaagcagttgtggcagaaattccggaagacaagccagaagctgctccagctcaaccaggcatgatgtaa

SEQ ID NO: 8 - DNA, 1962 bp, Lactobacillus delbrueckii strain DSM 33431
atggcagacgaagataacaaattgaagaaggctaagcagtttgaacaagaagctgacaagtacaatgccagccagattcaagtccttggcggcctggaagccgtacgcaagcgtccggggatgtacattggctcaaccagctcccagggcttgcaccacctggtttgggaaatcatcgacaacgggatcgacgaagccctggcaggtttcgcgaccaagatcgaagtcaccgtcaacgaagacaacagcgtgactgtccaagacgacgggcgggggatcccggtcgacatcgaaaagaagaccggccggccagccgttgaaactgtttatacggttctgcacgccggtggtaagttcggcggtggcggatacaaggtttccggcggtctgcacggggtcggggcttctgttgtcaacgccctgtccaccaagctgaacgtcactgtcatgcgggatggcaagaagtaccacatcgcctttgaccacggccgggtcgttgaagagctgaaggaagtcggcacggtgcctttgactgaacacgggaccatcgtccacttctggccggacccgaacatcttcacggaaaccacggtttttgacgacaagatcttgaagaaccggatccgggaactggccttcttgaacaaggggctgaagctgaccttcactgacaagcggaaggacacggcggaaaccgacgtctaccacttcgaaggtgggatcaaggaatacgtctccttcctcaaccggggccaggaggtactttttgacgagccaatctacgtggaaggcaagtacgagggcattgacgttgaagtatctttgcagtacacgaccggctacaagacaaccttgatgacttttgccaacaacatccacacttatgaaggcgggatgcacgaggcgggcttcaagacggccttgacccgggtggtcaacgactatgcccacaaggccaagatcttgaaggaaaacgatgacaacctgtccggggaagacatccgggaagggatgacggccgtgatttccgtcaagcacccgaacccgcagtttgaagggcagaccaagaccaagctgggcaactccgacgccagaactgccgttgaccgcgccttctcagaaaccttctccactttcttgatggaaaacccgcaggtggcccgcaagatcgttgaaaaggggcaactggccgaaagagccagagttgccgccaagcgcgcccgggaagttacccggaagaagtccggcctggaaatcgccaacctgccaggcaaattggccgacaacacttcaaatgacccgaacatctcagaactcttcatcgtcgaaggggactccgccggcggcagcgccaagcaagggcggagccggctgacccaggccatcctgcccatccgggggaagatcctgaacgtggaaaaggcctcaatggaccggatcctggccaaccaggaaatccggactctgtttacggccctggggaccggctttggggcagactttgacgtctccaaggcccgctatcacaagctgatcatcatgactgacgccgacgtcgacggggcccacatccggaccctgctcttgaccctcttctaccgctacatgcggccgatgatcgacaagggctatgtttacatcgcccgtccgcctctgtaccaagtgcgccagggcaagctgatcaagtacctggacactgatgaagaactccacgactacctggggtcattgcaaccaagcccgaagccaatagtgcagcgctacaaggggctgggggaaatggacgctgagcaattgtgggagacgaccatggatcctgaaaaccgccgcctggaccgggtggacccggaatacgccaaggacgcggatgaagtctttgaaatgctgatgggcaacgaagttggcccgcggcgcaagttcatcgaagacaatgcccaatatgttgaaaacttggatgcttaa

SEQ ID NO: 9 - DNA, 1365 bp, Lactobacillus delbrueckii strain DSM 33431
ttgttcgatttagaaaaattctgggatagcttcaacgctgaaatgcggtccgaatttaacgaagtatcctacaatgcctggttcaaaaacaccaagcccgtctccttcaacaaggacacccatgaattggtaatctccgtccaaacccccgtggcaaaggggtattgggagcaaaacatctccgctaacctgatccagtcggcttatgcctacgcgggaatcgatatctacccggttttcgtcgttaaaaacggcccggcgcccagcagcgaaagaatgctggagccccagccgcaggccaagcccgagaaagcccggccgcaaggccgcgaattcaccaaggacctgcgcttaaacgagaagtacaccttcgagaacttcattcaaggcgagggcaacaaactggctgccggggccgctttggcggttgccgacaacccggggaccttctacaaccccttgttcatcttcgggggcgtgggccttggtaaaacccacttgatgcaggcgatcggccaccagatgctggcggaaagaccagatgccaaggttgtctacattcaaagtgagacttttgtcaacgacttcatcaattccatcaagaacaagacccaggataagttccgggagaaataccggacagctgacctgctcttggtcgacgacatccagttcttcgccaagaaggaagggattcaggaagaattcttccatacttttgagactctctataatgatcaaaagcagatcgtcatgaccagtgaccggctgccaacggaaattcccgacctgtccgagcggctggtctcgcgttttgcctggggactgcaagttgaaatcaccccgccagacctggaaacccggatcgccatcctgcgcaagaaggctgaatccgaaggcctggagatcgatgaaagcaccctggactatgtggccagccaggtcgatactaatatccgggagctggaaggggccctggtcaaagtccaggcccaggccaccattcaaaagcaggacatcaacattggcttagcccggtcggctttagccgacctcaagctggtccagaagagcaggggactacagatttccaagatccaggaggtcgtggccaactacttccagacctcagtgccggaccttaaaggaaagaagcgggtaaggcaaatcgtcatcccgcgtcagattgccatgtacctgtcccgcgagctcaccgatgccagcctgccaaaaatcggccaggaattcgggggcaaggaccacacgaccgtgatgcatgcctgcgacaaaatcgcccggcagatcaaaacggacactgaaatcaagtccgctgtttccgacctgcggcagatgctggaaagatag

SEQ ID NO: 10 - DNA, 390 bp, Lactobacillus delbrueckii strain DSM 33431
atgcctgcaaataaaactgctcgtaagcgtcgtgtgaagaagcacgttgaaagtggcgtggctcacattcactcaacgtttaacaacactttagttatgattactgacgttcaaggtaacgccgtggcctggtcttcagctggtgctttgggtttcaagggtagccgtaagtctactccatttgctgcgcaaatggctgctgaagctgctgctaagagcgcaattgaccaaggtatgaagcacatcgaagtatcagttaagggcccaggttctggtcgtgaatcagcaattcgtgctctgcaagctgctggtctggaaatcacttcaatccgtgacgttactccagtacctcacaacggttctagaccaccaaagcgtcgtcgtgtttaa

SEQ ID NO: 11 - DNA, 186 bp, Lactobacillus delbrueckii strain DSM 33431
atggcaaaagatttcgtaaccggtaagaagaccacttttggtaacacgcgttcacacgctttgaactcatcaagtcgttcttggaagccaaaccttcaaaaagttcgcattcttgtaaacggcaagcctaagcgcgtttgggtttcaaccaagactttgaagtctggtaaagttacccgtgtctag

SEQ ID NO: 12 - DNA, 1561 bp, Lactobacillus delbrueckii strain DSM 33431
attgagagtttgatcctggctcaggacgaacgctggcggcgtgcctaatacatgcaagtcgagcgagctgaattcaaagatcccttcggggtgatttgttggacgctagcggcggatgggtgagtaacacgtgggcaatctgccctaaagactgggataccacttggaaacaggtgctaataccggataacaacatgaatcgcatgattcaagtttgaaaggcggcgcaagctgtcactttaggatgagcccgcggcgcattagctagttggtggggtaaaggcctaccaaggcaatgatgcgtagccgagttgagagactgatcggccacattgggactgagacacggcccaaactcctacgggaggcagcagtagggaatcttccacaatggacgcaagtctgatggagcaacgccgcgtgagtgaagaaggtcttcggatcgtaaagctctgttgttggtgaagaaggatagaggcagtaactggtctttatttgacggtaatcaaccagaaagtcacggctaactacgtgccagcagccgcggtaatacgtaggtggcaagcgttgtccggatttattgggcgtaaagcgagcgcaggcggaatgataagtctgatgtgaaagcccacggctcaaccgtggaactgcatcggaaactgtcattcttgagtgcagaagaggagagtggaactccatgtgtagcggtggaatgcgtagatatatggaagaacaccagtggcgaaggcggctctctggtctgcaactgacgctgaggctcgaaagcatgggtagcgaacaggattagataccctggtagtccatgccgtaaacgatgagcgctaggtgttggggactttccggtcctcagtgccgcagcaaacgcattaagcgctccgcctggggagtacgaccgcaaggttgaaactcaaaggaattgacgggggcccgcacaagcggtggagcatgtggtttaattcgaagcaacgcgaagaaccttaccaggtcttgacatcctgcgctacacctagagataggtggttcccttcggggacgcagagacaggtggtgcatggctgtcgtcagctcgtgtcgtgagatgttgggttaagtcccgcaacgagcgcaacccttgtctttagttgccatcattaagttgggcactctagagagactgccggtgacaaaccggaggaaggtggggatgacgtcaagtcatcatgccccttatgacctgggctacacacgtgctacaatgggcagtacaacgagaagcaaacccgcgagggtaagcggatctcttaaagctgctctcagttcggactgcaggctgcaactcgcctgcacgaagctggaatcgctagtaatcgcggatcagcacgccgcggtgaatacgttcccgggccttgtacacaccgcccgtcacaccatggaagtctgcaatgcccaaagtcggtgagataacctttataggagtcagccgcctaaggcagggcagatgactggggtgaagtcgtaacaaggtagccgtaggagaacctgcggctggatcacctccttt

SEQ ID NO: 13 - DNA, 1626 bp, Lactobacillus brevis strain DSM 33429
atggctaaagaattgaagttctctgaagacgcacgcagcaagatgcttgctggtgttgataaattagcgaacaccgttaagaccaccttaggacctaaggggcgtaacgtggttctcgaacaaagttatggcaacccaaccatcactaacgatggggtaacaattgctaaagcaattgaattagaagatcactttgaaaatatgggcgcaaagctcgtttccgaagttgcttccaagaccaacgatattgctggtgatgggaccactacggcgactgtattgacgcaagctatcgttaacgaaggcatgaagaacgttaccgctggggctaacccagtcggaatccgtcgtgggattgaaaaggcgactggtgccgctgttgatgctttgcacaagatgtctcatgacgttaagactaaagatgacatcgcccagattgcttccatttcttctgcaagtaaagaaactggtaagttaattgcggacgccatggaaaaggttggtaacgatggtgttatcaccattgaagaatcccgtggggttgatacgagcttagacgttgttgaagggatgcaattcgaccgtggttacatgtcacaatacatggtcacggataatgataagatggaagcaaaccttgacaacccatacatcttaatcacagacaagaagattgccaacatccaagacatcttgccattgttgcaatcagttgttgaacaaagccgttcactcttaatcatcgctgatgatatcacgggtgaagctttgccaaccttggtcttgaacaagatgcgtggaacgttcaacgttgtcgcagtcaaggcacctggctttggtgatcgtcgtaaggcacaattgcaagatatcgccgttttgactggtgggactgtgattaccgatgacttaggcttgaacctgaaggacacgacgattgatcaattgggtcaagcccaaaaggttaacgtgactaaggatgacaccacggtggttgaaggcgctggttctaaggaccaaatcgctgctcgcgtggctgaaatcaagggccaaattgaagacaccacgtctgactttgaccgtgataagctgaaggaacgtttagctaagttatctggtggtgttgctgtggttcgggtcggtgcagctactgaaaccgaattgaaggaacgcaagtaccggattgaagatgccctgaacgcgactcgagctgccgttgaagaaggcttcgttgctggtggtggtaccgcattgatcaatgttatcggtgacgttgctaagcttgaagctgaaggcgacgaaaagaccgggatcaacatcgttttacgtgctttggaagaacctgttcgtcaaatcgcacaaaacgccggtgttgaaggctctgttgtggttgaacacctgaagggtgaaaagccaggtgttggttacaacgctgctgacaacaagtacgaagacatggttgctgcgggaattaccgacccaactaaggtaacccgttctgctctgcaaaacgctgcttctgtatctgctttgttgttgacgacggaagccgttgtggctgaaaagccagaagacaatccagcaccagctgcaccagcagctaatccaggtatgggcggcatgatgtaa

SEQ ID NO: 14 - DNA, 1944 bp, Lactobacillus brevis strain DSM 33429
gtggcagacgaaaaagaaaccaaagcgcaacaagctcgtgagtatgacgccagtcaaattcaagtcttggaaggtttggaagcggtccggaaacggcccgggatgtacattggctcgacgagttcccaagggttacaccatttagtttgggaaattgtcgataacgggattgatgaggccttagcaggctttgctgatgaaatccacgtgacagtcaaccaagacaacagcattaccgttaccgataacggtcggggaattccgattggtatccaaaagaagacgggaaaaccagcgttagaaactgttttcacgattctgcatgccggtggtaaattcggcggtggcggatataaggtttctggtggtcttcatggggtgggtgcttccgtagttaacgccttgtctactgatcttgaggcgcaagtgacacgtgatggcaagacctatgcgattgcctttgaccacggccacgttaagacgccaatgcacgtgattaaagagggcctgccacaagaccaacacgggacggcagttcactttttaccggacccagatattttccgagaaacgaccgtctttgatattaagacattgacgacgcggattcgggaactggccttcttaaacaaaggcttgcggattactattcgtgatgaacgtccggaaaagccaacggaagaagatttcctttacgaaggcggtatccggcattacgtagactatttgaacgaaaacaaagaaacgttatttgaaccgccaatttacgtggaagggcaagaaaacggtattacggtggaagtcgcgttgcaatacacggacgactaccacaataacctcttaactttcaccaacaacatccacacctatgaaggtgggacccatgaagaagggttcaaacgggcgttgacgcgggttgtgaatgattatgcccggcaaaacaagttgatgaaagataatgaagccaacttgaccggggatgatgtccgtgaagggttgacagctgtggtttccgtgaagcatccggatccgcaatttgaaggtcaaacgaagaccaaattagggaactctgatgcccgcacggctgtcgatcacacctttgctgaccactttatgcggttcatgatggaacatccagatgtcgcccggaagattgtggacaaagggacgctggcgtctaaagcacgtgttgctgctaaacgggcccgtgaagtgacgcggaagaagagcggcctggaaatcagcaatttacctggtaagttagctgataacacgtctaaagatcccgaaatctctgaactctttatcgtcgaaggggattctgctggtgggtctgccaagcaaggtcgttcgcggctgacgcaggccattctgccaattcgggggaagattttaaacgtggaaaaggcgtcgatggaccggatcttagcaaacgaagaaattcggtcactctttacggctatgggaacgggttttggtaatgactttgatgtgtctaaggtaaattaccacaagcttattttgatgaccgatgccgatgtcgatggtgcgcacattcggacgctgttgttgacgctggtttaccgctacatgcggccattattggatgctggctttgtctatattgcctgcccaccgctttatcgagttcgccagggtaagatgcaacggtacttggattctgatgaagaattaaaggatctgttggggcaattgccaccatcaccaaaaccacaaatccaacggtataaagggttaggggaaatggacgcggaacaattgtgggaaacgaccatgaacccagaaaaccggcggctcttgcgggttgatgcgcaagatgccatcaatgccgatcaagtctttacgatgttgatgggcgataaggtggccccacggcgtgagtttatcgaaaataacgccacgtttgccgaactggatgtttag

SEQ ID NO: 15 - DNA, 1359 bp, Lactobacillus brevis strain DSM 33429
gtgccagacatgttaactttgtggactgatattaaggcgttgtttgaggaaaacaactcgaagacagcctacgcgacttggattgaaaccgccaagccgattgccttagatggcaacaaattaacgttggaactcccatcacccctgcatcgtgattactggacccatcaacacctggaccagcaattggtagaatacgcataccaagccgctcacgaagatattcaacccgtgttgattctcgaaaatgaacgtcagcaacaggccaccttaaaagccaaaaccgcacccgttgcggctggcgaaccggttgaaccgacacccacgtttatgaaagaaacggcgttgaattcccgctacacatttgacacctttgtcatcggaaaaggcaaccaaatggcccatgctgcggccttggtggtctctgaagaacccggcgtgatgtacaatccgctcttcttctatggtggcgttggtctgggcaagacccacttgatgcacgcgatcggcaacaaaatgttagaagatcgccccgacaccaaagttaaatacgtgactagcgaagccttcacgaatgactttattaatgcgattcaaactcggacgcaggagcagtttagacaggagtatcgtaacgttgatctgctgctggtcgatgatattcagttcttcgctaataaggaaggaacccaagaggagttcttccatacgtttaatgcgctctatgatgatggtaaacaaatcgtgctcacctcagatcgcttacccaacgagattcccaagctgcaagaccggttagtttctcggtttgcttggggattatccgttgatatcacaccgccagacctcgaaacgcgaattgctattttacggaacaaagccgacgccgatcagatcgacatccccgatgatacattaagctatatcgcgggccaaatcgactctaacgtccgtgaattagagggggctttagcccgcgttcaggcttattcgcaactcatgcaccaaccaattgccacggatttagctgctgaggcgctcaagagtctgaacctcgccaatgctagtgacgccgtgaccattcccgtgattcaagaccgcgtcgctaagtattttgacgtgtcgctcaaggatcttaaaggaaagaaacgcaagaaagccatcgttatgccgcgccaaatcgcgatgtacctctcccgtgaattaacggaagcctcactgccacggattggcaacgaatttggtggtaaagatcataccacggtgattcacgcttacgataaaattacggaatcccttaaaacggatccacaacttcaaaaagatatcgacagcctcaaagatgacttacgccgttaa

SEQ ID NO: 16 - DNA, 390 bp, Lactobacillus brevis strain DSM 33429
atggctactagaaaaactagtcgcaaacgtcgggtgaaaaagaacattgaagccggggttgcacatatccatgctacgtttaacaacactttggtcatgattactgacgttcaagggaacgcgatcgcttggtcatcagccggtgccttaggtttcaagggtagtcggaagtcaacaccatttgctgctcaaatggctgctgaagccgctgctaaagcatcacaagaacacggaattaagtccgttgaagttgctgttaagggtcctggttcaggacgtgaatctgcaattcgggccatccaagctgctgggatcgaaatcacggctattcgtgacgttaccccagtgcctcataatgggactcgtcctccaaagcgccgtcgggtttaa

SEQ ID NO: 17 - DNA, 186 bp, Lactobacillus brevis strain DSM 33429
atggcaaaggattttctcaacggcaagcggacgcacttcggtaacaagcgttctcacgctttgaattcaagtcgtcgcgcttggaagccgaacttacaaaaggttcgcatcttagtggacggtaagccaaagaaggtttggatttccgctcggactttgaaatcaggtaaagtaacccgcgtctag

SEQ ID NO: 18 - DNA, 1561 bp, Lactobacillus brevis strain DSM 33429
atgagagtttgatcctggctcaggacgaacgctggcggcatgcctaatacatgcaagtcgaacgagcttccgttgaatgacgtgcttgcactgatttcaacaatgaagcgagtggcgaactggtgagtaacacgtggggaatctgcccagaagcaggggataacacttggaaacaggtgctaataccgtataacaacaaaatccgcatggattttgtttgaaaggtggcttcggctatcacttctggatgatcccgcggcgtattagttagttggtgaggtaaaggcccaccaagacgatgatacgtagccgacctgagagggtaatcggccacattgggactgagacacggcccaaactcctacgggaggcagcagtagggaatcttccacaatggacgaaagtctgatggagcaatgccgcgtgagtgaagaagggtttcggctcgtaaaactctgttgttaaagaagaacacctttgagagtaactgttcaagggttgacggtatttaaccagaaagccacggctaactacgtgccagcagccgcggtaatacgtaggtggcaagcgttgtccggatttattgggcgtaaagcgagcgcaggcggttttttaagtctgatgtgaaagccttcggcttaaccggagaagtgcatcggaaactgggagacttgagtgcagaagaggacagtggaactccatgtgtagcggtggaatgcgtagatatatggaagaacaccagtggcgaaggcggctgtctagtctgtaactgacgctgaggctcgaaagcatgggtagcgaacaggattagataccctggtagtccatgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagctacgcgaagaaccttaccaggtcttgacatcttctgccaatcttagagataagacgttcccttcggggacagaatgacaggtggtgcatggttgtcgtcagctcgtgtcgtgagatgttgggttaagtcccgcaacgagcgcaacccttattatcagttgccagcattcagttgggcactctggtgagactgccggtgacaaaccggaggaaggtggggatgacgtcaaatcatcatgccccttatgacctgggctacacacgtgctacaatggacggtacaacgagttgcgaagtcgtgaggctaagctaatctcttaaagccgttctcagttcggattgtaggctgcaactcgcctacatgaagttggaatcgctagtaatcgcggatcagcatgccgcggtgaatacgttcccgggccttgtacacaccgcccgtcacaccatgagagtttgtaacacccaaagccggtgagataaccttcgggagtcagccgtctaaggtgggacagatgattagggtgaagtcgtaacaaggtagccgtaggagaacctgcggctggatcacctcctt

SEQ ID NO: 19 - DNA, 1626 bp, Lactobacillus plantarum subspecies argentoratensis DSM 33449
atggctaaagaattaaagttctctgaagatgcacgttcagcgatgctaaaaggtgtcgatcaattagctgacacagttaagtcaacgttaggtcctaagggtcgcaacgttgttttggaacaatcatatggttcaccaacaattactaatgatggtgtaacgattgctaaggcgatcgaattagacgatcatttcgaaaacatgggtgctaagttagtttctgaagttgcttcaaagactaatgacatcgctggtgatgggacgactactgcaacggtcttaacacaatcaatcgttaatgaaggtatgaagaacgttacggccggtgctaaccctgttggcattcgtcgtgggattgaagaagctactaagacggcggttgactcattacacgctatggcacacgaagttaagacgcaagaagatattgcgcaaatcgcttctgtatcttcagcaagtgaagaaactggtaaattgattgccgaagccatggaaaaagttggtcatgacggtgttatcacgattgaagaatcacgtggtgttgatactagcttagacgttgttgaagggatgcaattcgaccgcggctacttatcacaatacatggttactgataatgataagatggaagcggatcttgacaatccatatatcttaattactgataagaagatttcaaacattcaagatatcttaccactattacaatccatcgttgaacaaggcaagccattgttgatcattgctgatgacatttctggtgaagctttaccaaccttagtcttgaacaagatgcgtgggacgtttaacgttgtcgccgttaaggcacccggttttggtgatcggcgtaaggaacaattacaagatatcgctatcttaactggcgggacggttatcactgacgaccttggccttgaattgaaggacacgaccatcgatcaattaggtcaagccaacaaagttacggttactaaggataacaccaccattgttgaaggcgctggttccaaggatgctatctcagaacgggttgaatttatccgtaaccaaatcggtgaaacaacttctgactttgacaaagaaaagttacaagaacgtttagctaaattagctggtggggttgccgttgttcgtgtcggtgccgctactgaaactgaattgaaggaacgtaaataccggattgaagatgctttgaacgcaactcgggccgccgttgaagaaggctttgttgctggtggtggtactgctttgattaacgttatcaaagatgttgctgcattgaaggaaactggtgacgttcaaactgggatcaacattgttaaacgtgctttggaagaaccagttcgccaaatcgctgaaaatgctggtttagaaggctctgttatcgttgaaaagatgaaggaacaaaagccaggtgttggtttcaacgccgcaactgatgaatgggttgacatgatcaaagctggtatcgtggacccaactaaggtaacgcgttctgctttacaaaatgccgcttctgtttcagcccttctcttaacgactgaagccgttgtcgctgaaaaacctgaagaaaatgcaccagctgcaccagccgcaccaaacccaggtatgggcggtatgatgtaa

SEQ ID NO: 20 - DNA, 1947 bp, Lactobacillus plantarum subspecies argentoratensis DSM 33449
ttgaccgaaaacgaaaaagaaaccaaacttgaacaggctcgcgaatatgatgctagtcaaattcaagttttggaaggtttagaagctgtccggaaacgtcccggaatgtatatcggtacgacgagtagccaaggactccaccacttagtttgggaaattgttgataacggaattgatgaagccctagcagggtttgcaaacgtgatccacgtgaccgttgaaaaggataatagtattacggttactgataacggtcgtgggattcctgttgatattcaagaaaaaacgggtaaaccagcgcttgaaacggtttatacgattttacatgccggtggtaaattcggtggtggcggttataaagtctctggtggtcttcacggggtcggggcttccgttgttaacgccttatcatcgaccctggacgtcaaagtaactcggaatggtcatgtttattacatggactttgaacgtggaaaggttaaaacaccgatgaagatcattggtgatgcgcctgatgatgctcatggtacggcggttcattttgttccggatcctgatattttccgggaaacgaccacctatgacattaatattttgacgactcggattcgcgaactcgctttcttaaataagggattacggatcacgattcgggataatcgccctgatgaacccactgaagatgactttctctacgaaggtgggattcgtcactatgttgaatatctagataagaataagaccgttctatttccagaaccgatttatgtggaaggggaacagaatgggattacggtagaagttgcgttgcaatatactgacgactaccacagtaatttgatgaccttcaccaacaatattcatacgtacgaaggtgggacgcatgaagaaggtttcaaacgtgctttgacgcgggtcatcaacgactatgctcgtaagaataacttgttgaaggaaagcgatgcgaacctatctggcgaagatgttcgtgaagggatgacggctgttgtcagcgtcaagcatccggatcctcaatttgaaggtcaaacgaagacgaaacttggtaattccgatgctcgggcagcagttgaccggttattctcagaacactttagcaagtacttgatggaaaacccaagtgttgcccgtaaagtggtcgacaaagggttacttgcatccaaggcccgggtcgctgctaagcgtgcgcgggaagttacgcgaaaaaagagtggcttggaaattagtaatttaccaggtaagttggcggacaatagtagtaaagaccctgaaattagtgagttgttcattgtcgaaggggattctgccggtgggtcggctaagcagggacgttcccggttgacgcaagcgattttaccgattcggggtaaaattttgaacgttgaaaaggccagcattgataagattttagccaatgaagaaatccggtcattgtttacggcaatgggaactggcttcggtggtgacttcgacttgagcaaagccaattatcacaaattaattatcatgactgatgccgatgtcgatggggcacatattcggaccttgttattgacgttgttttaccggtacatgcggccattagttgacgctggctttgtgtacatcgctcaaccaccgttgtaccaagtccggcaaggtaaatttgtgaagtatattgattcagatgaagaattgagtgaggtcatggggcaattggcaccatcaccgaaaccagttgttcaacggtacaaggggcttggtgaaatggacgcggaacaattatgggaaacgaccatggatccagacaagcggcgcctattacgggttcgtgatgaagacgctgcggatgccgatggggtcttctcgatgttgatgggggaccaagtcaaaccacggcgagaatttattgaagataatgccaagttcgtccaggatctggatgtttag

SEQ ID NO: 21 - DNA, 1368 bp, Lactobacillus plantarum subspecies argentoratensis DSM 33449
gtgctggaacgtaatgacttatggaatctcattaagttctctatggaacaagatctgtccaagatcacgtttcaaacgtttgtcgaaccggcaaaaccactccaactcgataaagcccaaatgacgatcgaagtcccaacacaacttcatcgtgactattgggagaaaaacttggctgcaaagttcacggacattgcgatgcaagcgactaacgagcagattcggccggtcatgataacggaagaagaacgtcagcaacttacgagagataaggactcgcaagtgactaccggtaacgttgcgggacaacaaccaacaaccgcaactacccccacatttatgcgggaaacgaaactcaacccgaaatacacttttgatactttcgtgatcggtaaaggcaatcaaatggcccatgccgctgcgttagttgtgtcggaagaacccggcaccatgtataatccgttgtttttctacgggggcgttggtctgggaaaaacccacctaatgcacgctatcggtaacaaattgttagaaaccgatccgactagtaacattaaatatgtgactagcgaatcttttacgaatgaattaattaatgccattcaaactaaaaaacaggaggcgttccgcgaagaatatcggaacgttgacctgttattagtcgacgacattcaattttttgccaataaggaagcaacccaagaagagttcttccatacatttaatgctttatatgaagatgataagcaaatcgtgcttacatccgatcgcttaccgaacgaaattccgcaactccaagatcgcctagtttctaggtttaagtggggattatccgttgatattaccccacctgatctcgagacgcgaattgctattttgcgcaataaagccgatcttgaagggatcgaaatccctgacgatacgttaagttatatcgccggtcagatcgactcaaacgtgcgagaactcgaaggtgccttagcacgagtgcaggcatactcacgactgaataattcaccgattaccaccagtttagtggcggatgcattgaagagtttgcaccttagcagtaaactatcagaggtttcgattcccgtcattcaggaaaaagtcgccaagtacttccatgtaacaatggctgaccttaagggaaagaaacgcaataaacaaatcgtgattccacgtcaaattgccatgtatttatcacgcgaactgactgaaagttcacttccgcgcatcggtaacgagtttggtggtaaggaccatacgactgtgatccacgcccatgataaaatcactaaggcactcaaaactgatgctgaattacaaaaagccgtgggtgatttgacaggccagctcaaatcttag

SEQ ID NO: 22 - DNA, 390 bp, Lactobacillus plantarum subspecies argentoratensis DSM 33449; identical to SEQ ID NO: 4

SEQ ID NO: 23 - DNA, 186 bp, Lactobacillus plantarum subspecies argentoratensis DSM 33449; identical to SEQ ID NO: 5

SEQ ID NO: 24 - DNA, 1563 bp, Lactobacillus plantarum subspecies argentoratensis DSM 33449
tttgagagtttgatcctggctcaggacgaacgctggcggcgtgcctaatacatgcaagtcgaacgaactctggtattgattggtgcttgcatcatgatttacatttgagtgagtggcgaactggtgagtaacacgtgggaaacctgcccagaagcgggggataacacctggaaacagatgctaataccgcataacaacttggaccgcatggtccgagtttgaaagatggcttcggctatcacttttggatggtcccgcggcgtattagctagatggtgaggtaacggctcaccatggcaatgatacgtagccgacctgagagggtaatcggccacattgggactgagacacggcccaaactcctacgggaggcagcagtagggaatcttccacaatggacgaaagtctgatggagcaacgccgcgtgagtgaagaagggtttcggctcgtaaaactctgttgttaaagaagaacatatctgagagtaactgttcaggtattgacggtatttaaccagaaagccacggctaactacgtgccagcagccgcggtaatacgtaggtggcaagcgttgtccggatttattgggcgtaaagcgagcgcaggcggttttttaagtctgatgtgaaagccttcggctcaaccgaagaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagtatgggtagcaaacaggattagataccctggtagtccataccgtaaacgatgaatgctaagtgttggagggtttccgcccttcagtgctgcagctaacgcattaagcattccgcctggggagtacggccgcaaggctgaaactcaaaggaattgacgggggcccgcacaagcggtggagcatgtggtttaattcgaagctacgcgaagaaccttaccaggtcttgacatactatgcaaatctaagagattagacgttcccttcggggacatggatacaggtggtgcatggttgtcgtcagctcgtgtcgtgagatgttgggttaagtcccgcaacgagcgcaacccttattatcagttgccagcattaagttgggcactctggtgagactgccggtgacaaaccggaggaaggtggggatgacgtcaaatcatcatgccccttatgacctgggctacacacgtgctacaatggatggtacaacgagttgcgaactcgcgagagtaagctaatctcttaaagccattctcagttcggattgtaggctgcaactcgcctacatgaagtcggaatcgctagtaatcgcggatcagcatgccgcggtgaatacgttcccgggccttgtacacaccgcccgtcacaccatgagagtttgtaacacccaaagtcggtggggtaaccttttaggaaccagccgcctaaggtgggacagatgattagggtgaagtcgtaacaaggtagccgtaggagaacctgcggctggatcacctcctt